(19) United States
(12) Patent Application Publication
(10) Pub. No.: US 2026/0117186 A1
(43) Pub. Date: Apr. 30, 2026

(54) ADDITIVE COMPOSITIONS FOR CULTURE MEDIA, CULTURE MEDIA COMPRISING THE SAME AND METHODS OF CULTURING CELLS

(71) Applicant: NUTRIR LIMITED, Hong Kong (CN)

(72) Inventor: Yu Ka CHAN, Hong Kong (CN)

(21) Appl. No.: 19/125,741

(22) PCT Filed: Nov. 1, 2023
(86) PCT No.: PCT/CN2023/128948

§ 371 Date: Apr. 30, 2025

Related U.S. Application Data
(60) Provisional application No. 63/381,761, filed on Nov. 1, 2022.

(51) Int. Cl. C12N 5/077

(52) U.S. Cl. CPC C12N 5/0658; C12N 5/0656; C12N 5/0659; C12N 2500/74; C12N 2500/76; C12N 2500/80; C12N 2501/115; C12N 2501/15

(57) ABSTRACT

Provided is an additive composition for culture media, comprising a first component comprising at least one sample extract prepared from at least one natural substance, wherein the at least one natural substance is selected from a group consisting of bird's nest (BN), ginseng, Ling Zhi, versicolor mushroom, Cordyceps sinensis (CS), Ashwagandha (ASH), and combinations thereof. The additive composition can be acted as an alternative source of growth factors and hormones for cell culture media to improve consumer acceptance and/or to minimize the production costs.

### CROSS-REFERENCE TO RELATED APPLICATION

This application claims priority to, and the benefits of, U.S. Provisional Application having Ser. No. 63/381,761 filed on Nov. 1, 2022. The entire contents of the foregoing application are hereby incorporated by reference in its entirety for all purposes.

### FIELD OF INVENTION

This application relates to compositions, media, kits, methods and uses for culture media. In particular, this application relates to additive compositions, media, kits, methods and uses for culture media.

### BACKGROUND OF INVENTION

Growth factors and hormones are essential to cell-culture media (e.g., for culturing animal cells for producing animal tissues). For example, Fetal Bovine Serum (FBS) is widely used in cell-culture as a source of growth factors and hormones for culture media. However, FBS is highly unethical and expensive. Attempts have been made to produce recombinant growth factors and hormones to lower the cost. Overuse of recombinant growth factors can cause significant health concerns in consumers and cause consumer rejection in cell-cultured food products such as in cultured-meat. There is a great need to provide alternative culture medium additive compositions that are all-natural or largely natural, as an alternative source of growth factors and hormones for cell culture media to improve consumer acceptance and/or to minimize the production costs.

### SUMMARY OF INVENTION

In some embodiments, provided is an additive composition for a culture medium, including a first component including at least one sample extract prepared from at least one natural substance, wherein the at least one natural substance is selected from a group consisting of bird's nest (BN), ginseng, Ling Zhi (Ganoderma lucidum), versicolor mushroom, Cordyceps sinensis (CS), Ashwagandha (ASH), and combinations thereof.

In some embodiments, provided is an additive composition for a culture medium for culturing myoblasts, the additive composition includes: a first component, including at least one sample extract prepared from at least one natural substance, wherein the at least one natural substance is selected from a group consisting of bird's nest (BN), American Ginseng (AGN) and Korean Ginseng (KGN), and combination thereof, wherein individual sample extract has a final concentration of about 1-500 ug/mL in the culture medium.

In some embodiments, provided is an additive composition for a culture medium for culturing porcine skeletal muscle cells, the additive composition includes: a first component, including at least one sample extract prepared from at least one natural substance, wherein the at least one natural substance is selected from a group consisting of bird's nest (BN), American Ginseng (AGN) and Korean Ginseng (KGN), and combination thereof, wherein individual sample extract has a final concentration of about 1-500 ug/mL in the culture medium.

In some embodiments, provided is an additive composition for a culture medium for culturing bovine skeletal muscle cells, the additive composition includes: a first component, including at least one sample extract prepared from at least one natural substance, wherein the at least one natural substance is selected from a group consisting of bird's nest (BN), ginseng, Ling Zhi (Ganoderma Lucidum), versicolor mushroom, Cordyceps sinensis (CS), Ashwagandha (ASH), and combinations thereof, wherein individual sample extract has a final concentration of about 1-500 ug/mL in the culture medium.

In some embodiments, provided is a culture medium, including a basal medium or a minimal medium and the culture medium additive composition as described in any one of the preceding embodiments.

In some embodiments, provided is a method of culturing cells, which includes the following steps: (i) providing a basal medium or minimal medium and the culture medium additive composition as described in any one of the preceding embodiments to form a culture medium; and (ii) treating and culturing cells with the culture medium.

### ADVANTAGES OF THE PRESENT DISCLOSURE

In certain embodiments, provided additive compositions contain one or more extracts from biological or natural (such as non-mammals, plants or fungal) sources (such as bird's nests, and ginsengs, etc.) are used to either regulate growth factor production in cells, stabilize those growth factor for increased and longer activity, or regulate the same pathways that certain growth factors would. In certain embodiments, the extracts can also increase cell proliferation, increase cell stemness, inhibit cell differentiation, and/or induce cell differentiation. In addition to this, these extracts can provide additional health benefits, and from a traditional medicine's viewpoint (such as Traditional Chinese Medicine (TCM) or Ayurvedic Medicine), bring balance to both the cells in culture and consumers' bodies when consumed.

In certain embodiments, bird's nest is edible bird's nest made by swiftlets.

In certain embodiments, provided is alternatives/novel compositions, media, kits, methods and uses to minimize or even replace the use of one or more of the main and most expensive ingredients in cell-culture media for growing cells-growth factors, such as Fibroblast Growth Factor 2 (FGF-2) (such as Basic Fibroblast Growth Factor, bGFG) and TGF-b1. In certain embodiments, these alternatives can be derived from natural sources such as non-mammal/plant/fungi extracts, and they work by either being a homolog (with protein sequence that may be similar to those of growth factors with same the functions), regulating growth factor production in cells, regulating the same pathways as certain growth factors, and/or stabilizing the growth factors to be more potent. In certain embodiments, they can also serve as alternatives to hormones (e.g., insulin), or transferrins, having antimicrobial properties (e.g., prevent contamination in scale-up), having anti-cancer properties, and/or promoting extracellular matrix such as elastin and collagen production in the case of a skin care serum. Using these alternatives, exogenous growth factors, hormones, and antibiotics may not have to be added during cell-culture, which in turn minimize the cost and safety concern. In certain embodiments, they can also serve as alternatives to serums such as fetal bovine serum to produce additive compositions for serum-free, substantially serum-free or reduced-serum culture media. The production cost is significantly reduced.

In certain embodiments, using these high-quality, natural extracts can make it more appealing to consumers, and can be very advantageous in a marketing standpoint.

In certain embodiments, the provided novel compositions, media, kits, methods and uses are more consumer-friendly and more natural than using external or recombinant growth factors. In certain embodiments, they can also achieve the same goal of lowering costs of cultured-meat to reach (or be close to or even lower) price parity with regular meat.

In certain embodiments, the provided novel compositions, media, kits, methods and uses also reduce concerns during regulatory approval as these ingredients have a long history of being consumed without any substantial health/safety issues.

In certain embodiments, the provided novel compositions, media, kits, methods and uses can produce cells, cell cultures, cell tissues in various applications, such as but not limited to gene therapy, microfluidics, pharmaceuticals, nutraceuticals, diagnostics, transplantation, biotechnology etc.

In certain embodiments, the provided novel compositions, media, kits, methods and uses can also be nutritiously healthier, as some of these ingredients can prevent, treat diseases (such as cancer or diabetes), disorders or symptoms.

In certain embodiments, provided additive compositions contain one or more extracts that increase final cell yield via promoting cell proliferation and stemness by inhibiting differentiation. For example, in certain embodiments, stemness in skeletal muscle satellite cells can be increased such as via the regulation of Pax7 gene.

In certain embodiments, provided additive compositions contain one or more extracts that promote cell differentiation, for example, making muscle fibers or adipocytes mature.

In certain embodiments, provided additive compositions contain extracts that are used in place of growth factors such as vascular endothelial growth factors (VEGFs) and/or Fibroblast Growth Factors (FGFs), partially or entirely. In other words, the amount of exogeneous growth factors in the additive composition is reduced.

In certain embodiments, provided additive compositions contain extracts that are used in place of serums such as FBS, partially or entirely. In other words, the amount of exogeneous serums in the additive composition is reduced.

In certain embodiments, provided is alternatives/novel additive compositions, media, kits, methods and uses to regulate, mimic, stabilize, minimize or even replace the use of one or more of growth factors, hormones and/or serums. In certain embodiments, provided is alternatives/novel additive compositions, media, kits, methods and uses to regulate one or more pathways, i.e., have same or similar effects as growth factors, hormones and/or serums.

In certain embodiments, the growth factors are Fibroblast Growth Factor 2 (FGF-2) (such as Basic Fibroblast Growth Factor, bGFG), TGF-b1, VEGF, IGF, Insulin, EGF, PDGF, and/or IL-6.

In certain embodiments, the provided novel compositions, media, kits, methods and uses are used for producing cultured-meat. In certain embodiments, the cultured-meat are edible and safe for human or animal consumption.

In certain embodiments, the provided novel compositions, media, kits, methods and uses reduce costs of producing cells during cell culture such as in cultured-meat.

In certain embodiments, the provided novel compositions, media, kits, methods and uses showed that sample extracts from Panax ginseng (KGN), Panax quinquefolius (AGN), Ashwagandha (ASH), Cordyceps sinensis (CS), Edible Bird's Nest (BN), Coriolus versicolor (YZ), and Ganoderma lucidum (GL) have the potential to stimulate proliferation of various types of cells (such as L6, primary porcine myoblasts, bovine skeletal muscle fibroblasts) at various conditions such as reduced-serum/reduced-growth-factor culture media and serum-free/growth-factor-free culture media.

In certain embodiments, provided additive compositions contains multiple sample extracts at specific combinations and effective concentrations/ranges that provide unexpected synergistic effects to the culture medium with one or more advantages described above.

### BRIEF DESCRIPTION OF FIGURES

FIG. 1 is a plot showing the cell growth of porcine myoblasts under the stimulation of different example additive compositions containing various sample extracts (provided at 300 ug/mL) at the 56 h time point, according to an example embodiment.

FIG. 2 is a plot showing the cell growth of Bovine fibroblasts under the stimulation of different example additive compositions containing various sample extracts (provided at 160 ug/mL) at the 48 h time point, according to an example embodiment.

FIGS. 3A-3B are plots showing the cell growth of L6 myoblasts under the stimulation of example additive compositions containing sample extracts, Bird's Nest Extract or Panax ginseng Extract, at different concentrations at the 48 h time point, according to an example embodiment.

### DETAILED DESCRIPTION

Although the description referred to particular embodiments, the disclosure should not be construed as limited to the embodiments set forth herein.

As used herein and in the claims, the terms “comprising” (or any related form such as “comprise” and “comprises”), “including” (or any related forms such as “include” or “includes”), “containing” (or any related forms such as “contain” or “contains”), means including the following elements but not excluding others. It shall be understood that for every embodiment in which the term “comprising” (or any related form such as “comprise” and “comprises”), “including” (or any related forms such as “include” or “includes”), or “containing” (or any related forms such as “contain” or “contains”) is used, this disclosure/application also includes alternate embodiments where the term “comprising”, “including,” or “containing,” is replaced with “consisting essentially of” or “consisting of”. These alternate embodiments that use “consisting of” or “consisting essentially of” are understood to be narrower embodiments of the “comprising”, “including,” or “containing,” embodiments.

For the sake of clarity, “comprising”, “including”, “containing” and “having”, and any related forms are open-ended terms which allows for additional elements or features beyond the named essential elements, whereas “consisting of” is a closed end term that is limited to the elements recited in the claim and excludes any element, step, or ingredient not specified in the claim.

“Consisting essentially of” limits the scope of a claim to the specified materials, components, or steps (“essential elements”) that do not materially affect the essential characteristic(s) of the claimed invention. In some embodiments, the essential characteristics are the basic and novel characteristic(s) of the claimed invention.

As used herein, the singular forms “a”, “an” and “the” are intended to include the plural forms as well, unless the context clearly indicates otherwise. Where a range is referred in the specification, the range is understood to include each discrete point within the range. For example, 1-7 means 1, 2, 3, 4, 5, 6, and 7.

As used herein, the term “about” is understood as within a range of normal tolerance in the art and not more than +20% of a stated value. By way of example only, about 50 means from 40 to 60 including all values in between. As used herein, the phrase “about” a specific value also includes the specific value, for example, about 50 includes 50.

As used herein and in the claims, the terms “general” or “generally”, or “substantial” or “substantially” mean that the recited characteristic, angle, shape, state, structure, or value need not be achieved exactly, but that deviations or variations, including for example, tolerances, measurement error, measurement accuracy limitations and other factors known to those of skill in the art, may occur in amounts that do not preclude the effect the characteristic was intended to provide. For example, a composition that is “substantially” serum-free or a composition that “substantially does not comprise” would mean that the composition comprises either exactly no serum, nearly exactly no serum (e.g., below-detection-limit amount), or below 1% of the total composition.

As used herein, the terms “cultured-meat”, “cultivated-meat”, “in-vitro meat”, “cell-based meat”, “clean meat”, “synthetic meat”, and “lab-grown meat” are used interchangeably referring to a meat product comprising tissues derived from cell culturing. In some examples, cultured-meat is produced by culturing animal cells or tissues in vitro without growing the entire animal (also known as ‘cellular agriculture’). For example, the process involves isolating meat-specific cells from an animal, an expansion step where cells are grown in a nutrient-rich medium for cells to proliferate, followed by final harvesting of cells into the final meat product.

As used herein, the terms “proliferation” or “expansion” refers to the process of increasing cell number and cell population by culturing the desired cells.

As used herein, the term “culture medium” refers to a composition to support growth and/or other cellular activities of cells or cell tissues.

As used herein, the term “basal medium” or “minimal medium” refers to a growth medium that provides essential nutrients for survival and cultivation of desired cells. Basal medium may serve as a starting point or foundation upon which specific additives can be added to meet the specific requirements of the desired cell culture. Examples thereof include but not limited to Dulbecco's Modified Eagle Medium (DMEM), Ham's F12 medium (F12), Dulbecco's Modified Eagle Medium F12 (DMEM:F12), Ham's F10 medium (F10), Roswell Park Memorial Institute 1640 Medium (RPMI 1640), Minimum Essential Medium (MEM), serum free media (SFM), or combination thereof.

As used herein, the term “additive composition” refers to a formulation containing one or more additives or ingredients to meet one or more specific requirement(s) of a cell culture. Additive composition can be added into a basal medium to form a culture medium.

As used herein, the term “serum” refers to fluid component of blood that may include substances such as growth factors and hormones required for cell growth such as proliferation and/or differentiation. In some examples, serum such as fetal bovine serum are exogenous additive to the culture medium.

As used herein, the terms “growth factor” and “hormone” refer to substance such as protein which regulates signaling pathways and/or stimulate cell functions such as cell growth, proliferation and/or differentiation. Examples of growth factors or hormones include fibroblast growth factor (FGF) such as FGF-2, transforming growth factor (TGF) such as TGF-b1 and TGF-3, vascular endothelial growth factor (VEGF) such as VEGF-A, insulin-like growth factor (IGF) such as IGF-1, Insulin, epidermal growth factor (EGF), platelet-derived growth factor (PDGF) such as PDGF-BB, hepatocyte growth factor (HGF) and interleukin (IL) such as interleukin 6 (IL-6).

As used herein, the term “natural substance” refers to a material that is derived from naturally occurring sources (such as animals, fungi and plants) and is not significantly altered or artificially/chemically synthesized. In some examples, natural substance is a medicinal substance such as a Traditional Medicine, a Traditional Chinese Medicine (TCM) and/or a Ayurvedic Medicine. In some embodiments, the natural substance is derived from plants and fungi which contains a diverse range of proteins, polysaccharides, or other bioactives.

As used herein, the term “sample extract” refers to a portion or representative sample that has been extracted or prepared from a natural substance. In some examples, the sample extract is prepared by preparing a sample solution of the natural substance in a powder form.

As used herein, the term “bird's nest” or “edible bird's nest” refers to a natural substance made primarily from solidified saliva of swiftlets and is generally regarded as a TCM.

As used herein, the term “ginseng” refers to a perennial plant belonging to the genus Panax. In some examples, only the root portion is used. For example, Korean, Japanese or Chinese ginseng (Panax ginseng), and American ginseng (Panax quinquefolius) are two of the example ginseng species used as TCMs. Other ginseng species may be used. In some examples, ginseng from other locations such as Korean Peninsula, Northeast China, Russian Far East, Canada, Vietnam and the United States may be used. In some embodiments, Korean ginseng includes Korean Red Ginseng.

As used herein, the term “Ling Zhi” or “Lingzhi” refers to a polypore fungus belonging to the genus Ganoderma and is regarded as a TCM. For example, Ganoderma lucidum and Ganoderma sichuanense are two of the example Ling Zhi species used as TCMs.

As used herein, the term “versicolor mushroom” refers to a polypore mushroom belonging to the genus Trametes. For example, Coriolus versicolor (or Trametes versicolor) is one of the example versicolor mushroom species used as TCMs and it may also be called Yun Zhi.

As used herein, the term “Cordyceps sinensis” or “Ophiocordyceps sinensis” refers to an entomopathogenic fungus in the family of Ophiocordycipitaceae, and it is generally regarded as a TCM.

As used herein, the term “Ashwagandha” or “Withania somnifera” refers to a herb belonging to the genus Withania and is generally regarded as an Ayurvedic medicine.

As used herein, the term “exogenous” refers to some components or elements that originate from outside or external to the composition. For clarity's sake, if a component such as growth factors from a serum, chemically synthesized or recombinant growth factor or hormone is added to the additive composition, it is regarded as exogenous. On the other hand, if a growth factor or hormone is naturally present in or derived from the sample extract of a natural substance, it is not regarded as exogenous.

As used herein, the term “final concentration” refers to the concentration when a substance or a component (such as the first or second component) is present in a solution or a mixture (such as culture medium) after all necessary dilutions or reactions have taken place. In some examples, the total concentration of the culture medium is calculated in weight by volume (100% (w/v)). In some other examples, the total concentration of the culture medium is calculated in volume by volume (100% (v/v)).

As used herein, the term “animals” refers to eukaryote species such as mammalians (such as porcine, bovine, ovine, equine, canine, feline, rodent), birds or avian, reptile, fish, amphibians, crustaceans, mollusk, cephalopods or the like. etc.

### EMBODIMENTS OF THE PRESENT INVENTION

### Embodiment 1

In certain embodiments, the provided novel compositions, media, kits, methods and uses comprising extracts of a sample selected from a group consisting of bird's nests, ginsengs, Ashwagandha, Ganoderma lucidum, versicolor mushrooms, Cordyceps sinensis, and combination thereof, which have been validated to increase cell number, which are useful as additives to basal cell culture media such as DMEM media (or another in-house formulated basal media with necessary salts, buffers, minerals, amino acids, lipids, minerals etc.) for (e.g., scale-up) purposes of producing meat tissues.

In certain embodiments, the provided novel compositions, media, kits, methods and uses contains one or more of the following features:
1. Using natural extracts (in certain embodiments, Edible Bird's Nest, Korean Ginseng, and American Ginseng are used), the novel compositions are more consumer friendly, healthy, natural, and can potentially reduce costs.
2. Without being bound to any theory, these extracts may contain active ingredients to upregulate specific growth factors or hormones, and/or they may have homologous protein structures to the necessary growth factors or hormones. Therefore, they fully or partially replace the need for unnatural growth factor or hormone sources (e.g., recombinant sources) and also can reduce costs of producing cell culture such as cultured-meat.
3. Specific compounds from the extracts can be further isolated and/or purified for use instead of the entire extracts.
4. The extracts can be added to cell-culture media to replace the function of certain growth factors or hormones.
5. These extracts are used to retain meat cell stemness in culture. Larger cell yields can be obtained, such as by delaying uncontrolled cell differentiation.
6. These extracts are used to increase the proliferation rate of cells in culture. Larger cell yields can be obtained at a shorter time period.

In certain embodiments, provided is a culture medium additive composition, comprising at least one sample extract prepared from at least one natural substance. In certain embodiments, the at least one natural substance is a sample selected from a group consisting of bird's nests, ginsengs, Ashwagandha, Ganoderma lucidum, versicolor mushrooms, Cordyceps Sinensis, and combination thereof. In some embodiments, the ginseng is American Ginseng or Korean Ginseng. Other embodiments are described hereinafter.

In certain embodiments, the composition substantially does not comprise one or more exogenous growth factors or hormones, or combination thereof.

In certain embodiments, the at least one sample extract is bird's nest. In certain embodiments, the at least one sample extract is American Ginseng. In certain embodiments, the at least one sample extract is Korean Ginseng. In certain embodiments, the at least one sample extract is Ganoderma lucidum. In certain embodiments, the at least one sample extract is versicolor mushroom. In certain embodiments, the at least one sample extract is Coriolus versicolor. In certain embodiments, the at least one sample extract is Cordyceps Sinensis. In certain embodiments, the at least one sample extract is Ashwaganda. In certain embodiments, the Ashwaganda is derived from a root only or a whole plant.

In certain embodiments, the sample extract is prepared from a solution of a filtrate or a supernatant. In certain embodiments, the solution comprises water. In certain embodiments, the solution comprises an organic solvent.

In certain embodiments, the composition essentially consists of at least one sample extract prepared from at least one sample selected from a group consisting of bird's nests, ginsengs, Ganoderma lucidum, versicolor mushrooms, Cordyceps sinensis, Ashwagandha, and combination thereof.

In certain embodiments, the at least one sample extract comprises a concentration of 0.001 ug/mL-1000 mg/mL. In certain embodiments, the at least one sample extract comprises a concentration of 20 mg/mL-100 mg/mL. In certain embodiments, the at least one sample extract comprises a concentration of 20 mg/mL. In certain embodiments, the at least one sample extract comprises a concentration of 60 mg/mL. In certain embodiments, the at least one sample extract comprises a concentration of 100 mg/mL. In certain embodiments, the sample extract is prepared from an aqueous solution of a filtrate or a supernatant.

In certain embodiments, provided is a culture medium, comprising a basal medium or a minimal medium and the culture media additive composition as disclosed in any one of the embodiments above.

In certain embodiments, the culture medium substantially does not comprise one or more exogenous growth factors or hormones, or combination thereof. In certain embodiments, the culture medium further comprises one or more exogenous growth factors or hormones, or combination thereof.

In certain embodiments, the basal medium is Dulbecco's modified eagle medium (DMEM) and/or F12.

In certain embodiments, the composition further comprises FBS at a concentration of 5%.

In certain embodiments, provided is a method of culturing cells, comprising the following steps: (i) providing a culture medium comprising a basal medium and at least one sample extract prepared from at least one natural substance; and (ii) treating and culturing cells with the at least one natural substance. In certain embodiments, the at least one natural substance is a sample consisting of bird's nests, ginsengs, Ashwagandha, Ling Zhi (e.g., Ganoderma lucidum), versicolor mushrooms (such as Yun Zhi), Cordyceps sinensis, and combination thereof. In certain embodiments, the method further comprises step of (1) treating a cell line with a detachment agent (e.g., trypsin) to form a cell suspension, after step (ii). In certain embodiments, the method further comprises step of (2) neutralizing the cell suspension with a neutralizing agent (e.g., a trypsin neutralizing solution, TrypLE) to obtain a neutralized cell suspension, prior to step (ii). In certain embodiments, the method further comprises step of (3) obtaining a cell pellet from the neutralized cell suspension and resuspending the cell pellet in the culture medium.

In certain embodiments, the sample extract is prepared by the following steps: (a) Preparing at least one powder of the at least one sample; (b) Dissolving the at least one powder to form at least one sample solution; (c) Purifying the at least one sample solution to obtain the at least one sample extract. In certain embodiments, the sample extract is prepared by the following steps: (a) preparing at least one powder of the at least one sample; (b) dissolving the at least one powder into water to form at least one sample solution; (c) filtering and/or centrifuging the at least one sample solution to obtain the at least one sample extract.

In certain embodiments, the culture medium comprises a final sample extract concentration of about 0.001 ug/mL-1000 mg/mL. In certain embodiments, the culture medium comprises a final sample extract concentration of about 0.1 mg/mL-0.5 mg/mL. In certain embodiments, the culture medium comprises a final sample extract concentration of about 0.1 mg/mL. In certain embodiments, the culture medium comprises a final sample extract concentration of about 0.3 mg/mL. In certain embodiments, the culture medium comprises a final sample extract concentration of about 0.5 mg/mL.

In certain embodiments, the culturing cells are for producing animal tissues. In certain embodiments, the animal tissues are human tissues. In certain embodiments, the culturing cells are for producing cultured-meat.

In certain embodiments, the at least one sample solution comprises a solvent. In certain embodiments, the at least one sample solution comprises water. In certain embodiments, the at least one sample solution comprises an organic solvent.

### Embodiment 2

In some embodiments, provided is an additive composition for a culture medium, including a first component including at least one sample extract prepared from at least one natural substance, wherein the at least one natural substance is selected from a group consisting of bird's nest (BN), ginseng, Ling Zhi, versicolor mushroom, Cordyceps sinensis (CS), Ashwagandha (ASH), and combinations thereof.

In some embodiments, the ginseng is Panax quinquefolius, Panax ginseng, and combinations thereof.

In some embodiments, the versicolor mushroom is Yun Zhi (YZ).

In some embodiments, the Ling Zhi is Ganoderma lucidum (GL).

In some embodiments, the versicolor mushroom is Coriolus versicolor.

In some embodiments, the at least one natural substance is bird's nest.

In some embodiments, the at least one natural substance is American Ginseng (Panax quinquefolius).

In some embodiments, the at least one natural substance is Korean Ginseng (Panax ginseng).

In some embodiments, the at least natural substance is Ganoderma lucidum.

In some embodiments, the at least one natural substance is versicolor mushroom.

In some embodiments, the at least one natural substance is Cordyceps sinensis.

In some embodiments, the at least one natural substance is Ashwaganda.

In some embodiments, the Ashwaganda is derived from a root or a whole plant.

In some embodiments, the first component substantially does not include one or more exogenous serums, growth factors or hormones, or combinations thereof.

In some embodiments, the culture medium is for culturing animal cells.

In some embodiments, the culture medium is for culturing mammalian, avian, crustaceans or fish cells.

In some embodiments, the culture medium is for culturing fibroblasts, vascular cells, pericytes, endothelial cells, myoblast cells, skeletal muscle satellite cells, muscle cells, smooth muscle cells, fat cells, adipose-derived stem cells, skin cells, tendon, liver, brain, bone, heart, kidney, stem cells, or combination thereof.

In some embodiments, the culture medium is for culturing fibroblasts or myoblasts.

In some embodiments, the individual sample extract is prepared from a solution of a filtrate or a supernatant of the individual natural substance.

In some embodiments, the first component essentially consists of at least one sample extract prepared from at least one natural substance, wherein the at least one natural substance is selected from a group consisting of bird's nests, ginsengs, Ganoderma lucidum, versicolor mushrooms, Cordyceps sinensis, Ashwagandha, and combination thereof.

In some embodiments, the individual sample extract has a final concentration of about 0.01 ng/mL-1000 mg/mL in the culture medium.

In some embodiments, the individual sample extract has a final concentration of about less than 1000 or 500 ug/mL in the culture medium.

In some embodiments, the additive composition further includes a second component including at least one serum, growth factor or hormone, amino acid, vitamin, salt, minerals, glucose, buffering agent, or combinations thereof.

In some embodiments, the second component further includes fetal bovine serum (FBS) at a final concentration of less than about 20%, 10%, 5% or 2% in the culture medium.

In some embodiments, the second component further includes bovine serum albumin (BSA) at a final concentration of about 0.01 ng/ml-10 mg mL in the culture medium.

In some embodiments, the second component further includes bovine serum albumin (BSA) at a final concentration of less than about 10, 9, 8, 7, 6, 5, 4, 3, 2, or 1 mg/mL in the culture medium.

In some embodiments, the at least one growth factor or hormone is FGF-2, TGF-b1, vascular endothelial growth factor (VEGF), insulin-like growth factor (IGF), Insulin, EGF, platelet-derived growth factor (PDGF), interleukin 6 (IL-6) or combinations thereof.

In some embodiments, the second component further includes TGF-b1 is present at a final concentration of less than about 10, 9, 8, 7, 6, 5, 4, 3, 2, 1 or 0.5 ng/ml in the culture medium.

In some embodiments, the second component further includes fetal bovine serum (FBS), fetal calf serum, bovine serum albumin (BSA), or combinations thereof.

In some embodiments, provided is an additive composition for a culture medium for culturing myoblasts, the additive composition includes: a first component, including at least one sample extract prepared from at least one natural substance, wherein the at least one natural substance is selected from a group consisting of bird's nest (BN), American Ginseng (AGN) and Korean Ginseng (KGN), and combination thereof, wherein individual sample extract has a final concentration of about 1-500 ug/mL in the culture medium.

In some embodiments, the additive composition further includes a second component including FBS having a final concentration of about 2-5% in the culture medium.

In some embodiments, provided is an additive composition for a culture medium for culturing porcine skeletal muscle cells, the additive composition includes: a first component, including at least one sample extract prepared from at least one natural substance, wherein the at least one natural substance is selected from a group consisting of bird's nest (BN), American Ginseng (AGN) and Korean Ginseng (KGN), and combination thereof, wherein individual sample extract has a final concentration of about 1-500 ug/mL in the culture medium.

In some embodiments, the additive composition further includes a second component including FBS having a final concentration of about 2-5% in the culture medium.

In some embodiments, the additive composition further includes a second component including BSA having a final concentration of about 1-10 mg/ml and optionally TGF-b1 having a final concentration of about 0.5-5 ng/ml in the culture medium.

In some embodiments, provided is an additive composition for a culture medium for culturing bovine skeletal muscle cells, the additive composition includes: a first component, including at least one sample extract prepared from at least one natural substance, wherein the at least one natural substance is selected from a group consisting of bird's nest (BN), ginseng, Ling Zhi (Ganoderma Lucidum), versicolor mushroom, Cordyceps sinensis (CS), Ashwagandha (ASH), and combinations thereof, wherein individual sample extract has a final concentration of about 1-500 ug/mL in the culture medium.

In some embodiments, the additive composition further includes a second component including FBS having a final concentration of about 2-5% in the culture medium.

In some embodiments, provided is a culture medium, including a basal medium or a minimal medium and the culture medium additive composition as described in any one of the preceding embodiments.

In some embodiments, the culture medium substantially does not include or include a reduced amount of one or more exogenous serums, growth factors or hormones, or combinations thereof.

In some embodiments, the basal medium or minimal medium is Dulbecco's Modified Eagle Medium (DMEM), Ham's F12 medium (F12), Dulbecco's Modified Eagle Medium F12 (DMEM:F12), Ham's F10 medium (F10), Roswell Park Memorial Institute 1640 Medium (RPMI 1640), Minimum Essential Medium (MEM), serum free medium (SFM) or combination thereof.

In some embodiments, provided is a method of culturing cells, including the following steps: (i) providing a basal medium or minimal medium and the culture medium additive composition as described in any one of the preceding embodiments to form a culture medium; and (ii) treating and culturing cells with the culture medium.

In some embodiments, the method further includes a step of: (1) treating a cell line with a detachment agent to form a cell suspension.

In some embodiments, the method further includes steps of: (2) neutralizing the cell suspension with a neutralizing agent to obtain a neutralized cell suspension.

In some embodiments, the method further includes step of: (3) obtaining a cell pellet from the neutralized cell suspension and resuspending the cell pellet in the culture medium.

In some embodiments, the sample extract is prepared by one or more of the following steps: (a) preparing at least one powder of the at least one natural substance; (b) dissolving the at least one powder to form at least one sample solution; (c) purifying the at least one sample solution to obtain the at least one sample extract.

In some embodiments, the purifying the at least one sample solution in step (c) is filtering and/or centrifuging the at least one sample solution.

In some embodiments, the culturing cells are for producing animal tissues.

In some embodiments, the animal tissues are mammalian, avian, crustaceans or fish tissues.

In some embodiments, the culturing cells are for producing cultured-meat.

In some embodiments, the method further includes a step of incubating the cells in a CO2 incubator.

In some embodiments, the cell line is derived from mammals, birds, crustaceans or fish.

In some embodiments, the cell line is derived from fibroblasts, vascular cells, pericytes, endothelial cells, myoblast cells, satellite cells, skeletal muscle cells, muscle cells, smooth muscle cells, fat cells, adipose-derived stem cells, skin cells, tendon, liver, brain, bone, heart, kidney, stem cells, and combination thereof.

In some embodiments, the cell line is derived from mammals, birds, crustaceans or fish. In some embodiments, the cell line is derived from Crustaceans (such as shrimps), Aves (such as chicken), Actinopterygii (such as pond loach, salmon), or Mammalia (such as bovine or porcine).

In some embodiments, the cell line is derived from muscle cells (such as skeletal muscle cells, smooth muscle cells), myoblast cells, skeletal muscle satellite cells, fat cells, adipose-derived stem cells, fibroblasts, vascular cells (such as endothelial cells, pericytes), skin cells, tendon, liver, brain, bone, heart, kidney, stem cells, and combination thereof. In some embodiments, the cell line is derived from skeletal muscle cells, satellites and myoblasts, and/or cells arising therefrom (e.g. myocytes, muscle fibers). In some embodiments, the cell line is derived from adipose cells, such as adipose derived stem cells (aka pre-adipocytes), mature adipocytes, and/or cells arising therefrom. In some embodiments, the cell line is derived from fibroblast cells. In some embodiments, the cell line is derived from induced pluripotent stem cells (iPSCs).

In some embodiments, the additive composition and the basal medium together form a culture medium with a total concentration of 100%.

In some embodiments, the additive composition comprises a first component comprising one or more sample extracts prepared from one or more natural substances as described herein, wherein individual or each sample extract has a final concentration of about 0-99% (or more) in the culture medium. For example, the appropriate concentration ranges are any ranges between 0-99%, such as 0.00001-1%, 0.0001-0.1%, 0.1-5%, 0.01-10%, 0.001-20%, 1-30%, 5-40%, 10-50%, 20-60%, 30-70%, 40-80%, or more. In some embodiments, the individual or each sample extract has a concentration of about 0.00005%, about 0.0001%, about 0.0002%, about 0.0003%, about 0.0004%, about 0.0005%, about 0.0006%, about 0.0007%, about 0.0008%, about 0.0009%, about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1.0%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%. In some further embodiments, individual or each sample extract has a final concentration of 0.01 ng mL-1000 mg/mL (or more) in the culture medium. In some further embodiments, the individual or each sample extract has a concentration of about 0.01 ng/ml-100 ng/mL. In some further embodiments, the individual or each sample extract has a concentration of about 0.01 ug/mL-100 ug/mL. In some further embodiments, the individual or each sample extract has a concentration of about 0.01 mg/mL-100 mg/mL. In some further embodiments, the individual or each sample extract has a concentration of about 0.2 mg/mL-100 mg/mL. In some further embodiments, individual or each sample extract has a first component at a concentration of about 10 ug/mL-1000 ug/mL. In some further embodiments, individual or each sample extract has a concentration of about 0.01 ng/ml, 0.02 ng/mL, 0.03 ng/mL, 0.04 ng/mL, 0.05 ng/mL, 0.06 ng/ml, 0.07 ng/ml, 0.08 ng/ml, 0.09 ng/ml, 0.1 ng/mL, 0.2 ng/ml, 0.3 ng/mL, 0.4 ng/mL, 0.5 ng/mL, 0.6 ng/mL, 0.7 ng/mL, 0.8 ng/mL, 0.9 ng/ml, 1 ng/mL, 2 ng/ml, 3 ng/ml, 4 ng/ml, 5 ng/ml, 6 ng/ml, 7 ng/ml, 8 ng mL, 9 ng/mL, 10 ng/mL, 11 ng/mL, 12 ng/ml, 13 ng/ml, 14 ng/ml, 15 ng/mL, 16 ng/ml, 17 ng/mL, 18 ng/ml, 19 ng/ml, 20 ng/ml, 21 ng/mL, 22 ng/ml, 23 ng/mL, 24 ng/mL, 25 ng/mL, 26 ng/ml, 27 ng/ml, 28 ng/ml, 29 ng/ml, 30 ng/ml, 31 ng/mL, 32 ng/mL, 33 ng/mL, 34 ng/ml, 35 ng/ml, 36 ng/ml, 37 ng/ml, 38 ng/ml, 39 ng/mL, 40 ng/mL, 41 ng/mL, 42 ng/mL, 43 ng/mL, 44 ng/ml, 45 ng/ml, 46 ng/ml, 47 ng/mL, 48 ng/mL, 49 ng/mL, 50 ng/mL, 51 ng/mL, 52 ng/ml, 53 ng/ml, 54 ng/mL, 55 ng/mL, 56 ng/mL, 57 ng/mL, 58 ng/ml, 59 ng/ml, 60 ng/ml, 61 ng/ml, 62 ng/ml, 63 ng/mL, 64 ng/mL, 65 ng/mL, 66 ng/ml, 67 ng/ml, 68 ng/ml, 69 ng/ml, 70 ng/mL, 71 ng/mL, 72 ng/mL, 73 ng/mL, 74 ng/mL, 75 ng/mL, 76 ng/ml, 77 ng/ml, 78 ng/ml, 79 ng/mL, 80 ng/mL, 81 ng/mL, 82 ng/mL, 83 ng/mL, 84 ng/mL, 85 ng/ml, 86 ng/ml, 87 ng/mL, 88 ng/mL, 89 ng/mL, 90 ng/ml, 91 ng/ml, 92 ng/mL, 93 ng/mL, 94 ng/mL, 95 ng/ml, 96 ng/mL, 97 ng/mL, 98 ng/mL, 99 ng/ml, 0.1 ug/mL, 0.2 ug/mL, 0.3 ug/mL, 0.4 ug/mL, 0.5 ug/mL, 0.6 ug/mL, 0.7 ug/mL, 0.8 ug/mL, 0.9 ug/mL, 1 ug/mL, 2 ug/mL, 3 ug/mL, 4 ug/mL, 5 ug/mL, 6 ug/mL, 7 ug/mL, 8 ug/mL, 9 ug/mL, 10 ug/mL, 11 ug/mL, 12 ug/mL, 13 ug/mL, 14 ug/mL, 15 ug/mL, 16 ug/mL, 17 ug/mL, 18 ug/mL, 19 ug/mL, 20 ug/mL, 21 ug/mL, 22 ug/mL, 23 ug/mL, 24 ug/mL, 25 ug/mL, 26 ug/mL, 27 ug/mL, 28 ug/mL, 29 ug/mL, 30 ug/mL, 31 ug/mL, 32 ug/mL, 33 ug/mL, 34 ug/mL, 35 ug/mL, 36 ug/mL, 37 ug/mL, 38 ug/mL, 39 ug/mL, 40 ug/mL, 41 ug/mL, 42 ug/mL, 43 ug/mL, 44 ug/mL, 45 ug/mL, 46 ug/mL, 47 ug/mL, 48 ug/mL, 49 ug/mL, 50 ug/mL, 51 ug/mL, 52 ug/mL, 53 ug/mL, 54 ug/mL, 55 ug/mL, 56 ug/mL, 57 ug/mL, 58 ug/mL, 59 ug/mL, 60 ug/mL, 61 ug/mL, 62 ug/mL, 63 ug/mL, 64 ug/mL, 65 ug/mL, 66 ug/mL, 67 ug/mL, 68 ug/mL, 69 ug/mL, 70 ug/mL, 71 ug/mL, 72 ug/mL, 73 ug/mL, 74 ug/mL, 75 ug/mL, 76 ug/mL, 77 ug/mL, 78 ug/mL, 79 ug/mL, 80 ug/mL, 81 ug/mL, 82 ug/mL, 83 ug/mL, 84 ug/mL, 85 ug/mL, 86 ug/mL, 87 ug/mL, 88 ug/mL, 89 ug/mL, 90 ug/mL, 91 ug/mL, 92 ug/mL, 93 ug/mL, 94 ug/mL, 95 ug/mL, 96 ug/mL, 97 ug/mL, 98 ug/mL, 99 ug/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, 200 mg/mL, 210 mg/mL, 220 mg/mL, 230 mg/mL, 240 mg/mL, 250 mg/mL, 260 mg/mL, 270 mg/mL, 280 mg/mL, 290 mg/mL, 300 mg/mL, 310 mg/mL, 320 mg/mL, 330 mg/mL, 340 mg/mL, 350 mg/mL, 360 mg/mL, 370 mg/mL, 380 mg/mL, 390 mg/mL, 400 mg/mL, 410 mg/mL, 420 mg/mL, 430 mg/mL, 440 mg/mL, 450 mg/mL, 460 mg/mL, 470 mg/mL, 480 mg/mL, 490 mg/mL, 500 mg/mL, 510 mg/mL, 520 mg/mL, 530 mg/mL, 540 mg/mL, 550 mg/mL, 560 mg/mL, 570 mg/mL, 580 mg/mL, 590 mg/mL, 600 mg/mL, 610 mg/mL, 620 mg/mL, 630 mg/mL, 640 mg/mL, 650 mg/mL, 660 mg/mL, 670 mg/mL, 680 mg/mL, 690 mg/mL, 700 mg/mL, 710 mg/mL, 720 mg/mL, 730 mg/mL, 740 mg/mL, 750 mg/mL, 760 mg/mL, 770 mg/mL, 780 mg/mL, 790 mg/mL, 800 mg/mL, 810 mg/mL, 820 mg/mL, 830 mg/mL, 840 mg/mL, 850 mg/mL, 860 mg/mL, 870 mg/mL, 880 mg/mL, 890 mg/mL, 900 mg/mL, 910 mg/mL, 920 mg/mL, 930 mg/mL, 940 mg/mL, 950 mg/mL, 960 mg/mL, 970 mg/mL, 980 mg/mL, 990 mg/mL, 1000 mg/mL, or more. In some embodiments, the additive composition comprises a first component at a concentration of about 0.2 mg/mL, 0.4 mg/mL, 0.8 mg/mL, 1.6 mg/mL, 3.2 mg/mL, 6.4 mg/mL, 20 mg/mL, 60 mg/mL, or 100 mg/mL. The one or more sample extracts are prepared from at least one natural substance, wherein the at least one natural substance is selected from a group consisting of bird's nest (BN), ginseng (such as American Ginseng (Panax quinquefolius), Korean or Chinese ginseng (Panax ginseng)), Ling Zhi (such as Ganoderma lucidum (GL)), versicolor mushroom (such as Yun Zhi (YZ), Cordyceps sinensis (CS), Ashwagandha (ASH), and combinations thereof.

In one further embodiment, the additive composition comprises or essentially consists of bird's nest at a concentration of about 0.01 ng/ml-1000 mg/mL, or any concentrations described above.

In one further embodiment, the additive composition comprises or essentially consists of ginseng such as American Ginseng or Panax quinquefolius at a concentration of about 0.01 ng/ml-1000 mg/mL, or any concentrations described above.

In one embodiment, the culture medium additive composition comprises or essentially consists of ginseng such as Korean Ginseng, Chinese Ginseng or Panax ginseng at a concentration of about 0.01 ng/ml-1000 mg/mL, or any concentrations described above.

In one embodiment, the additive composition comprises or essentially consists of versicolor mushroom such as Yun Zhi at a concentration of about 0.01 ng/ml-1000 mg/mL, or any concentrations described above.

In one embodiment, the additive composition comprises or essentially consists of Ling Zhi such as Ganoderma lucidum at a concentration of about 0.01 ng/ml-1000 mg/mL, or any concentrations described above.

In one embodiment, the additive composition comprises or essentially consists of Cordyceps sinensis at a concentration of about 0.01 ng/ml-1000 mg/mL, or any concentrations described above.

In one embodiment, the culture medium additive composition comprises or essentially consists of Ashwagandha at a concentration of about 0.01 ng/ml-1000 mg/mL, or any concentrations described above.

In some embodiments, the culture medium comprises a first component at a final concentration of about 0.01 ng/mL-1000 mg/mL, or any concentrations described above. In some embodiments, the culture medium comprises a first component at a final concentration of about 20 ug/mL-0.5 mg/mL. In some embodiments, the culture medium comprises a first component at a final concentration of about 1 ug/mL, 5 ug/mL, 10 ug/mL, 20 ug/mL, 40 ug/mL, 80 ug/mL, 100 ug/mL, 160 ug/mL, 300 ug/mL, or 500 ug/mL.

In one further embodiment, the culture medium comprises an additive composition comprising one or more sample extracts prepared from one or more natural substances as described herein, at a final concentration of about 0.01 ng/ml-1000 mg/mL, or any concentrations described above and a basal medium such as DMEM, or DMEM:F12.

In further embodiments, the culture medium optionally comprises a second component as described herein. In some further embodiments, the second component comprises one or more exogenous ingredients such as serums, growth factors, hormones, amino acids, vitamins, salts, glucose, buffering agents, or combinations thereof. Individual or each ingredient in the second component has reduced amounts or concentrations in the culture medium, such as a final concentration of about 0-99% (or more) in the culture medium. For example, any appropriate concentration ranges between 0-99%, such as 0.0001-50%, 1-20%, or 0.01 ng/ml-1000 mg/mL (or more) in the culture medium. In some further embodiments, the individual or each ingredient in the second component has a concentration of about 0.0000151-1%, 0.0001-0.1%, 0.1-5%, 0.01-10%, 0.001-20%, 0.01-10%, 0.1-5%, 1-30%, 5-40%, 10-50%, 20-60%, 30-70%, 40-80%, or more. In some embodiments, the individual or each sample extract has a concentration of about 0.00005%, about 0.0001%, about 0.0002%, about 0.0003%, about 0.0004%, about 0.0005%, about 0.0006%, about 0.0007%, about 0.0008%, about 0.0009%, about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1.0%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%. In some further embodiments, the individual or each ingredient in the second component has a concentration of about 0.01 ng/ml-100 ng/ml. In some further embodiments, the individual or each ingredient in the second component has a concentration of about 0.01 ug/mL-100 ug/mL. In some further embodiments, the individual or each ingredient in the second component has a concentration of about 0.01 mg/mL-100 mg/mL. In some further embodiments, the individual or each ingredient in the second component has a concentration of about 0.2 mg/mL-100 mg/mL. In some further embodiments, individual or each ingredient in the second component has a first component at a concentration of about 10 ug/mL-1000 ug/mL. In some further embodiments, individual or each ingredient in the second component has a concentration of about 0.01 ng/mL, 0.02 ng/mL, 0.03 ng/ml, 0.04 ng/mL, 0.05 ng/mL, 0.06 ng/mL, 0.07 ng/mL, 0.08 ng/mL, 0.09 ng/mL, 0.1 ng/mL, 0.2 ng/mL, 0.3 ng/mL, 0.4 ng/mL, 0.5 ng/mL, 0.6 ng/ml, 0.7 ng/ml, 0.8 ng/mL, 0.9 ng/mL, 1 ng/ml, 2 ng/ml, 3 ng/mL, 4 ng/mL, 5 ng/mL, 6 ng/ml, 7 ng/ml, 8 ng/ml, 9 ng/ml, 10 ng/mL, 11 ng/mL, 12 ng/mL, 13 ng/mL, 14 ng/mL, 15 ng/ml, 16 ng/ml, 17 ng/ml, 18 ng/mL, 19 ng/mL, 20 ng/mL, 21 ng/ml, 22 ng/ml, 23 ng/ml, 24 ng/ml, 25 ng/ml, 26 ng/mL, 27 ng/mL, 28 ng/mL, 29 ng/ml, 30 ng/mL, 31 ng/ml, 32 ng/ml, 33 ng/ml, 34 ng/mL, 35 ng/mL, 36 ng/mL, 37 ng/ml, 38 ng/mL, 39 ng/ml, 40 ng/ml, 41 ng/ml, 42 ng/mL, 43 ng/mL, 44 ng/mL, 45 ng/mL, 46 ng/ml, 47 ng/ml, 48 ng/ml, 49 ng/ml, 50 ng/mL, 51 ng/mL, 52 ng/mL, 53 ng/mL, 54 ng/ml, 55 ng/ml, 56 ng/ml, 57 ng/ml, 58 ng/mL, 59 ng/mL, 60 ng/mL, 61 ng/mL, 62 ng/mL, 63 ng/mL, 64 ng/ml, 65 ng/mL, 66 ng/mL, 67 ng/mL, 68 ng/mL, 69 ng/mL, 70 ng/ml, 71 ng/ml, 72 ng/mL, 73 ng/ml, 74 ng/mL, 75 ng/mL, 76 ng/mL, 77 ng/ml, 78 ng/mL, 79 ng/mL, 80 ng/mL, 81 ng/ml, 82 ng/mL, 83 ng/mL, 84 ng/mL, 85 ng/ml, 86 ng/ml, 87 ng/ml, 88 ng/ml, 89 ng/ml, 90 ng/ml, 91 ng/mL, 92 ng/mL, 93 ng/ml, 94 ng/ml, 95 ng/ml, 96 ng/ml, 97 ng/ml, 98 ng/ml, 99 ng/ml, 0.1 ug/mL, 0.2 ug/mL, 0.3 ug/mL, 0.4 ug/mL, 0.5 ug/mL, 0.6 ug/mL, 0.7 ug/mL, 0.8 ug/mL, 0.9 ug/mL, 1 ug/mL, 2 ug/mL, 3 ug/mL, 4 ug/mL, 5 ug/mL, 6 ug/mL, 7 ug/mL, 8 ug mL, 9 ug/mL, 10 ug/mL, 11 ug/mL, 12 ug/mL, 13 ug/mL, 14 ug/mL, 15 ug/mL, 16 ug/mL, 17 ug/mL, 18 ug/mL, 19 ug/mL, 20 ug/mL, 21 ug/mL, 22 ug/mL, 23 ug/mL, 24 ug/mL, 25 ug/mL, 26 ug/mL, 27 ug/mL, 28 ug/mL, 29 ug/mL, 30 ug/mL, 31 ug/mL, 32 ug/mL, 33 ug/mL, 34 ug/mL, 35 ug/mL, 36 ug/mL, 37 ug/mL, 38 ug/mL, 39 ug/mL, 40 ug/mL, 41 ug/mL, 42 ug/mL, 43 ug/mL, 44 ug/mL, 45 ug/mL, 46 ug/mL, 47 ug/mL, 48 ug/mL, 49 ug/mL, 50 ug/mL, 51 ug/mL, 52 ug/mL, 53 ug/mL, 54 ug/mL, 55 ug/mL, 56 ug/mL, 57 ug/mL, 58 ug/mL, 59 ug/mL, 60 ug/mL, 61 ug/mL, 62 ug/mL, 63 ug/mL, 64 ug/mL, 65 ug/mL, 66 ug/mL, 67 ug/mL, 68 ug/mL, 69 ug/mL, 70 ug/mL, 71 ug/mL, 72 ug/mL, 73 ug/mL, 74 ug/mL, 75 ug/mL, 76 ug/mL, 77 ug/mL, 78 ug/mL, 79 ug/mL, 80 ug/mL, 81 ug/mL, 82 ug/mL, 83 ug/mL, 84 ug/mL, 85 ug/mL, 86 ug/mL, 87 ug/mL, 88 ug/mL, 89 ug/mL, 90 ug/mL, 91 ug/mL, 92 ug/mL, 93 ug/mL, 94 ug/mL, 95 ug/mL, 96 ug/mL, 97 ug/mL, 98 ug/mL, 99 ug/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, 41 mg/mL, 42 mg/mL, 43 mg/mL, 44 mg/mL, 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, 55 mg/mL, 56 mg/mL, 57 mg/mL, 58 mg/mL, 59 mg/mL, 60 mg/mL, 61 mg/mL, 62 mg/mL, 63 mg/mL, 64 mg/mL, 65 mg/mL, 66 mg/mL, 67 mg/mL, 68 mg/mL, 69 mg/mL, 70 mg/mL, 71 mg/mL, 72 mg/mL, 73 mg/mL, 74 mg/mL, 75 mg/mL, 76 mg/mL, 77 mg/mL, 78 mg/mL, 79 mg/mL, 80 mg/mL, 81 mg/mL, 82 mg/mL, 83 mg/mL, 84 mg/mL, 85 mg/mL, 86 mg/mL, 87 mg/mL, 88 mg/mL, 89 mg/mL, 90 mg/mL, 91 mg/mL, 92 mg/mL, 93 mg/mL, 94 mg/mL, 95 mg/mL, 96 mg/mL, 97 mg/mL, 98 mg/mL, 99 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, 200 mg/mL, 210 mg/mL, 220 mg/mL, 230 mg/mL, 240 mg/mL, 250 mg/mL, 260 mg/mL, 270 mg/mL, 280 mg/mL, 290 mg/mL, 300 mg/mL, 310 mg/mL, 320 mg/mL, 330 mg/mL, 340 mg/mL, 350 mg/mL, 360 mg/mL, 370 mg/mL, 380 mg/mL, 390 mg/mL, 400 mg/mL, 410 mg/mL, 420 mg/mL, 430 mg/mL, 440 mg/mL, 450 mg/mL, 460 mg/mL, 470 mg/mL, 480 mg/mL, 490 mg/mL, 500 mg/mL, 510 mg/mL, 520 mg/mL, 530 mg/mL, 540 mg/mL, 550 mg/mL, 560 mg/mL, 570 mg/mL, 580 mg/mL, 590 mg/mL, 600 mg/mL, 610 mg/mL, 620 mg/mL, 630 mg/mL, 640 mg/mL, 650 mg/mL, 660 mg/mL, 670 mg/mL, 680 mg/mL, 690 mg/mL, 700 mg/mL, 710 mg/mL, 720 mg/mL, 730 mg/mL, 740 mg/mL, 750 mg/mL, 760 mg/mL, 770 mg/mL, 780 mg/mL, 790 mg/mL, 800 mg/mL, 810 mg/mL, 820 mg/mL, 830 mg/mL, 840 mg/mL, 850 mg/mL, 860 mg/mL, 870 mg/mL, 880 mg/mL, 890 mg/mL, 900 mg/mL, 910 mg/mL, 920 mg/mL, 930 mg/mL, 940 mg/mL, 950 mg/mL, 960 mg/mL, 970 mg/mL, 980 mg/mL, 990 mg/mL, 1000 mg/mL, or more. In some embodiments, the additive composition comprises a first component at a final concentration of about 1 ug/mL, 5 ug/mL, 10 ug/mL, 20 ug/mL, 40 ug/mL, 80 ug/mL, 100 ug/mL, 160 ug/mL, 300 ug/mL, or 500 ug/mL. In further embodiments, the second component comprises FBS at a final concentration of about 0.01-50% (such as 0.1-20%, or 2-5%) or more in the culture medium. In other embodiments, the second component comprises BSA at a final concentration of about 1-100 mg/mL (such as 6 mg/mL) and/or TGF-b1 at a final concentration of about 0.1-100 ng/ml (such as 2 ng/ml). The second component comprises at least one serum (such as FBS), growth factor or hormone (such as FGF-2, TGF-b1, vascular endothelial growth factor (VEGF), insulin-like growth factor (IGF), Insulin, EGF, platelet-derived growth factor (PDGF), interleukin 6 (IL-6)), amino acid (such as BSA), vitamin, salt, glucose, buffering agent, or combinations thereof.

### EXAMPLES

Provided herein are examples that describe in more detail certain embodiments of the present disclosure. The examples provided herein are merely for illustrative purposes and are not meant to limit the scope of the invention in any way. All references given below and elsewhere in the present application are hereby included by reference.

### Example 1

### Preparation of Example Sample Extracts

TABLE 1
Samples used in this Example embodiment

| Sam- | | | | |
|---|---|---|---|---|
| ple | | | Used | |
| no. | Sample Type | Form | Amount | Identity |
| 1 | Korean Ginseng | dried, herbal root | 500 mg | Panax ginseng |
| | (KG) | portion | | |
| 2 | American | dried, herbal root | 500 mg | Panax |
| | Ginseng (AG) | portion | | quinquefolius |
| 3 | bird's nest | dried, edible | 500 mg | Bird's nest made |
| | (BN) | bird's nest | | by swiftlets |

1. The samples were ground into a powder form (e.g., using an electric food chopper and then a coffee grinder).
2. Each sample was placed in a 50 mL tube, and 5 ml of water was added, respectively, allowing each of them to dissolve for 24 hours in a shaking incubator at 100 rpm. (Final concentration=500 mg/5 mL=100 mg/mL)
3. The samples were filtered through a sterile 0.2 um filter into a 15 ml tube. This step and the following steps were performed in a biosafety cabinet to ensure sterility. In case any one of the samples was unable to be filtered, the samples were centrifuged and then filtered to obtain the supernatants as sample extracts. Optionally, the samples were filtered through a 100 um cell strainer first prior to filtration.
4. Three 1.5 mL centrifuge tubes were prepared for each sample extract (triplicate).
5. Each sample extract was aliquoted into the centrifuge tubes and added water as needed to obtain the final concentrations as follows:
   a. Tube 1:100 mg/ml
   b. Tube 2:60 mg/ml
   c. Tube 3:20 mg/ml

### Testing Sample Extracts

1. In this example, example culture cells used for testing was L6 myoblasts cells (not bovine) which were cultured in DMEM supplemented with 10% FBS. Made sure to introduce serum starvation by replacing the media with DMEM ONLY media for 4 hours before the next steps. For positive control, the medium used was DMEM supplemented with 10% FBS.
2. The cells were rinsed twice more with PBS after 4 hours.
3. PBS was removed, and the cells were trypsinized. The cells were treated with trypsin (Gibco, 0.25% Trypsin-EDTA, phenol red) for 3 to 5 mins with routine tapping of the flasks to promote cell detachment.
4. Cell counting was performed once the trypsin was neutralized with TNS (trypsin neutralizing solution). The ratio of the volume of TNS to Trypsin for neutralization is 1:1.
5. The cells were centrifuged (RCF=150 g) into a pellet.
6. The cell pellet was resuspended in DMEM such that there were 2×10⁴ cells/mL. 500 uL of the cell suspension were added into each well for test in a 24 well plate.
7. Well arrangement:
   a. Column 1: Sample extract 1: Korean Ginseng
   b. Column 2: Sample extract 2: American Ginseng
   c. Column 3: Sample extract 3: Bird's Nest
   d. Row A (for Col. 1-3): 5 uL of extract was added from tube 1 to the well. Also added 495 uL of DMEM in 10% FBS.
   e. Row B (for Col. 1-3): Added 5 uL of extract from tube 2. Also added 495 uL of DMEM in 10% FBS.
   f. Row C (for Col. 1-3): Added 5 uL of extract from tube 3. Also added 495 uL of DMEM in 10% FBS.
   g. NEGATIVE CONTROL: Row D (for Col. 1-3): Added 500 uL of DMEM ONLY
   h. POSITIVE CONTROL 1: Column 4 Row 1-3 (Well 4A+4B+4C): Added 480 uL of DMEM. Add 20 uL of FGF-2 (from a 5 ug/mL FGF-2 aliquot) such that the final concentration of FGF-2 in the well is 0.1 ug/mL.
   i. ADDITIONAL POSITIVE CONTROL 2: Column 5 Row 1-3 (Wells 5A+5B+5C): Added 500 uL of DMEM+20% FBS. (Final concentration: 10% FBS)
8. Repeated steps 1-7 on another plate
   a. The first plate were cultured for 24 hours.
   b. The second plate were cultured for 48 hours.
9. Assess proliferation with cell counting by a hemocytometer using trypan blue as a staining agent.

### Results

### (1) 24 Hour Time Point

Samples were incubated with the cells in the first plate for 24 hours, before cell counting was performed.

TABLE 2
Summarized cell counts and cell viabilities of cells treated
with sample extracts for 24 hours (0.3 ml T/E + 0.3 ml
TNS = 0.6 ml total volume; initial cell number = 10,000 cells/well).

| Sample extract | Results | |
|---|---|---|
| Korean Ginseng | Cell Density Count 1 (cells/mL)= | 8.21 × 10⁴ |
| (100 ug/mL) + 5% | Viability 1= | 36% |
| FBS, DMEM | Cell Density Count 2 (cells/mL)= | 8.80 × 10⁴ |
| | Viability 2= | 40% |
| | Total cell count (cells)= | 5.10 × 10⁴ |
| | Average viability= | 38% |
| | Cell Number Increase= | 510% |
| American Ginseng | Cell Density Count 1 (cells/mL)= | 3.52 × 10⁴ |
| (100 ug/mL) + 5% | Viability 1= | 50% |
| FBS, DMEM | Cell Density Count 2 (cells/mL)= | 1.29 × 10⁵ |
| | Viability 2= | 18% |
| | Total cell count (cells)= | 4.93 × 10⁴ |
| | Average viability= | 25% |
| | Cell Number Increase= | 493% |
| Bird's Nest | Cell Density Count 1 (cells/mL)= | 1.06 × 10⁵ |
| (100 ug/mL) + 5% | Viability 1= | 94% |
| FBS, DMEM | Cell Density Count 2 (cells/mL)= | 8.21 × 10⁴ |
| | Viability 2= | 79% |
| | Total cell count (cells)= | 5.64 × 10⁴ |
| | Average viability= | 87% |
| | Cell Number Increase= | 564% |
| Negative Control | Cell Density Count 1 (cells/mL)= | 1.35 × 10⁵ |
| (DMEM only) | Viability 1= | 100% |
| | Cell Density Count 2 (cells/mL)= | 3.52 × 10⁴ |
| | Viability 2= | 50% |
| | Total cell count (cells)= | 5.11 × 10⁴ |
| | Average viability= | 50% |
| | Cell Number Increase= | 511% |
| Positive Control | Cell Density Count 1 (cells/mL)= | 1.52 × 10⁵ |
| (DMEM + 0.1 | Viability 1= | 77% |
| ug/mL FGF-2) | Cell Density Count 2 (cells/mL)= | 1.47 × 10⁵ |
| | Viability 2= | 96% |
| | Total cell count (cells)= | 8.97 × 10⁴ |
| | Average viability= | 86% |
| | Cell Number Increase= | 897% |
| 2^{nd} Positive Control | Cell Density Count 1 (cells/mL)= | 8.21 × 10⁴ |
| (DMEM + 10% FBS) | Viability 1= | 43% |
| | Cell Density Count 2 (cells/mL)= | 1.41 × 10⁵ |
| | Viability 2= | 63% |
| | Total cell count (cells)= | 6.69 × 10⁴ |
| | Average viability= | 56% |
| | Cell Number Increase= | 669% |

### (2) 48 Hour Time Point

Sample extracts were incubated cells for 48 hours, before cell counting was performed

TABLE 3
Summarized cell counts and cell viabilities of cells
treated with sample extracts for 48 hours (0.38 ml T/E + 0.38 ml
TNS = 0.76 ml total volume; initial cell number = 10,000 cells/well).

| Sample Extract | Results | |
|---|---|---|
| Korean Ginseng | Cell Density Count 1 (cells/mL)= | 3.11 × 10⁵ |
| (100 ug/mL) + 5% | Viability 1= | 15% |
| FBS, DMEM | Cell Density Count 2 (cells/mL)= | 3.23 × 10⁵ |
| | Viability 2= | 25% |
| | Total cell count (cells)= | 2.41 × 10⁵ |
| | Average viability= | 20% |
| | Cell Number Increase= | 2409% |
| American Ginseng | Cell Density Count 1 (cells/mL)= | 9.09 × 10⁵ |
| (100 ug/mL) + 5% | Viability 1= | 30% |
| FBS, DMEM | Cell Density Count 2 (cells/mL)= | 9.33 × 10⁵ |
| | Viability 2= | 25% |
| | Total cell count (cells)= | 7.00 × 10⁵ |
| | Average viability= | 27% |
| | Cell Number Increase= | 7000% |
| Bird's Nest | Cell Density Count 1 (cells/mL)= | 4.99 × 10⁵ |
| (100 ug/mL) + 5% | Viability 1= | 20% |
| FBS, DMEM | Cell Density Count 2 (cells/mL)= | 2.87 × 10⁵ |
| | Viability 2= | 24% |
| | Total cell count (cells)= | 2.99 × 10⁵ |
| | Average viability= | 21% |
| | Cell Number Increase= | 2987% |
| Negative Control | Cell Density Count 1 (cells/mL)= | 2.64 × 10⁵ |
| (DMEM only) | Viability 1= | 27% |
| | Cell Density Count 2 (cells/mL)= | 1.17 × 10⁵ |
| | Viability 2= | 35% |
| | Total cell count (cells)= | 1.45 × 10⁵ |
| | Average viability= | 29% |
| | Cell Number Increase= | 1448% |
| Positive Control | Cell Density Count 1 (cells/mL)= | 1.35 × 10⁵ |
| (DMEM + 0.1 | Viability 1= | 87% |
| ug/mL FGF-2) | Cell Density Count 2 (cells/mL)= | 1.17 × 10⁵ |
| | Viability 2= | 75% |
| | Total cell count (cells)= | 9.58 × 10⁴ |
| | Average viability= | 81% |
| | Cell Number Increase= | 958% |
| 2^{nd} Positive Control | Cell Density Count 1 (cells/mL)= | 2.46 × 10⁵ |
| (DMEM + 10% FBS) | Viability 1= | 40% |
| | Cell Density Count 2 (cells/mL)= | 1.99 × 10⁵ |
| | Viability 2= | 91% |
| | Total cell count (cells)= | 1.69 × 10⁵ |
| | Average viability= | 63% |
| | Cell Number Increase= | 1691% |

### Results and Discussions:

The above results showed that during the first 24 hour the cell number stays relatively the same as that of the negative control (DMEM only), suggested that the cells might not be upregulated by the samples yet. Only after 24-48 hours, the cell numbers treated with the three sample extracts surprisingly increase, indicating the sample extracts stimulate cell proliferation. Among the three sample extracts, Sample extract of American ginseng surprisingly outperforms the other two, followed by Bird's Nest and Korean ginseng. By time point 48 hours, all Sample extracts even surprisingly surpass the performances of the positive controls, including DMEM supplementing with 0.05 ug/ml FGF-2 and DMEM supplementing with 10% FBS.

In summary, bird's nest, American Ginseng (AG), and Korean Ginseng (KG) sample extracts were tested with their growth factor upregulation abilities. In 48-hour muscle cell-culture, a concentration of 100 ug/mL of KG and AG results in 24.09 and 70.00 times increase in cell numbers respectively, whereas 0.1 ug/mL of FGF-2 results in a 9.58 increase. If similar cell proliferation numbers are maintained even without the addition of 5% FBS, this translates to a dramatic (e.g., 22,285-fold, and 18,665-fold, respectively) cost reduction for the FGF-2 component for using KG, and AG as replacement, because in certain examples, FGF-2 is one of the most expensive components in cell-culturing.

The percentage (%) proliferation increases in 24 hours and 48 hours normalized to the negative control were summarized shown in Table 4. The % of proliferation increase after normalized to control is calculated as follows:

$$\% of proliferation increase = \{ [ ( Total cell count in samples ) - ( Total cell count in negative control with DMEM only ) ] / ( Total cell count negative control with DMEM only ) \} * 100 \%$$

TABLE 4
Cell proliferation increase (%) in 24 hours and 48 hours

% Proliferation Increase
(Normalized to negative
controls)

| | | |
|---|---|---|
| Sample Extract conc. and type | 24 h | 48 h |

| | | | |
|---|---|---|---|
| 100 ug/mL | KGN | −0.196% | 66.207% |
| | AGN | −3.523% | 382.759% |
| | BN | 10.372% | 106.207% |

The results in Table 4 showed that culture media containing basal medium DMEM, and example additive composition containing first component of KGN, AGN or BN at about 100 ug/ml and a second component of FBS at 5% in the final concentration had superior % proliferation increases (66.207%, 382.759% and 106.207%, respectively) at 48 hours. Results indicated that the example additive compositions containing these sample extracts (AGN, KGN, BN) have surprising effects in cell proliferation for cells such as L6 myoblasts. This example demonstrated that sample extracts can significantly reduce the serum (FBS) used in media from 10% to 5% in the final concentration. Sample extracts in the presence of 5% serum can surprisingly outperform even the positive control groups having 10% serum.

### Example 2

In this example, the effects on the proliferation of porcine skeletal muscle cells using the culture medium compositions containing various example additive compositions was demonstrated. In this example, the culture media were tested with porcine skeletal muscles cells. The culture media are also applicable to both myoblasts and skeletal muscle satellite cells.

Materials used in this example include:
Cell line: Porcine skeletal muscle cells (PIG-iCell-S005) (Cellverse Bioscience Technology Co., Ltd.)
Basal medium: Dulbecco's Modified Eagle Medium (DMEM) (Thermofisher, D6429)
Second component: Fetal Bovine Serum (ScienCell, 0500), Bovine Serum Albumin (BSA) (Sigma A7030-10g), TGF-b1 (Thermofisher, PHG9204), FGF-2 (Thermofisher, PHG0026)
Other reagents: 0.25% Trypsin/EDTA (Gibco, 25200-056), CyQuant MTT (Invitrogen, V13154) and Gelatin (Sigma G1890)

### Example Sample Extracts:

Edible Bird's Nest,
Panax ginseng (also referred to as ‘Korean Ginseng’ or ‘KGN’) (Jung Kwan Jang) extract and
Panax quinquefolius (also referred to as ‘American Ginseng’ or ‘AGN’) extract (President's Brand) were prepared in a similar manner as described in Example 1.

In this example, the steps below were carried out:
1. Stock solutions of sample extracts (AGN, KGN) at a concentration of about 1 mg/mL, 3 mg/mL, and 5 mg/mL were prepared.
2. Stock solutions of Bovine Serum Albumin (BSA) at a concentration of about 80 mg/mL was prepared in smaller volumes (<1 mL) in distilled water (H₂O). The high concentration of the BSA stock solution allows better maintenance of cell osmotic balance.
3. The following solutions were prepared:

$$a . M 1 Media : DMEM + 2 ng / mL TGF - b 1 + 1 mg / nL BSA + 40 ng / mL FGF - 2$$

$$b . M 3 Media : DMEM + 2 ng / mL TGF - b 1 + 1 mg / nL BSA$$

4. All the wells to be used in a 24 well plate were coated with 2% gelatin.
5. Porcine skeletal muscle cells in DMEM supplemented with about 10% FBS were cultured until a final cell concentration of at least 1×10⁶ cells/mL was achieved.
6. Appropriate volumes of about 0.25% Trypsin/EDTA were used to harvest porcine skeletal muscle cells, then neutralized with Trypsin Neutralizer Solution (also referred to as “neutralizing agent”), and centrifuged at 300 g for 5 minutes. The supernatant was discarded and resuspended cells in M3 media at a final cell density of about 1×10⁶ cells/mL.
7. About 10 uL of cell suspension were added in each of the wells to be used (Final Cell Density: about 1×10⁴ cells/well).
8. About 840 uL of M3 media were added, and cells were left for about 1 hour to allow for serum starvation. For the control groups with either M3 media (negative control), or M1 media (also referred as ‘positive control’ or ‘M1’), the same amounts of M3 or M1 medium were added, respectively.
9. About 50 uL of about 100 mg/mL BSA were added such that the final concentration in each well were about 5-6 mg/mL. The same was also performed for the controls.
10. About 100 uL of stock sample extracts were added in each well. For the controls, 100 uL phosphate buffered saline (PBS) was added instead of sample extracts. The concentrations of components of each culture medium are shown in Table 5 (KGN=Korean Ginseng, Panax ginseng, AGN=American Ginseng, Panax quinquefolius).

TABLE 5
Final concentrations of the example culture
medium compositions used in Example 2

| | Culture medium composition | | |
|---|---|---|---|
| | Additive composition | | |
| | First | Second | |
| Sample | Component | Component | Basal medium |
| A1 | 500 ug/mL AGN | 5.84 mg/mL BSA, | DMEM |
| | | 1.7 ng/mL TGF-b1 | |
| A2 | 300 ug/mL AGN | 5.84 mg/mL BSA, | DMEM |
| | | 1.7 ng/mL TGF-b1 | |
| A3 | 100 ug/mL AGN | 5.84 mg/mL BSA, | DMEM |
| | | 1.7 ng/mL TGF-b1 | |
| B1 | 500 ug/mL KGN | 5.84 mg/mL BSA, | DMEM |
| | | 1.7 ng/mL TGF-b1 | |
| B2 | 300 ug/mL KGN | 5.84 mg/mL BSA, | DMEM |
| | | 1.7 ng/mL TGF-b1 | |
| B3 | 100 ug/mL KGN | 5.84 mg/mL BSA, | DMEM |
| | | 1.7 ng/mL TGF-b1 | |
| Positive | — | 5.84 mg/mL BSA, | DMEM |
| control | | 1.7 ng/mL TGF-b1, | |
| (M1) | | 34 ng/mL FGF-2 | |
| Negative | — | 5.84 mg/mL BSA, | DMEM |
| control | | 0.02 ng/mL TGF-b1 | |

11. Step 10 was repeated as needed to obtain replicates. In this example, two replicates for each well were obtained.
12. After about 56 hours, media from wells were transferred to individual centrifuge tubes. When spent media was removed, unattached cells are removed as well. Adhered cells remained on the well plate were extracted with the following steps:
13. 2 mL of 0.25% Trypsin/EDTA were then added into each well and incubated for 15-30 min. Gentle tapping on the sides and bottom of the well was conducted.
14. 20 uL of detached cells in 0.25% Trypsin/EDTA were mixed with 20 uL of Trypan Blue.
15. 15 uL of the detached cells and trypan blue mix was added to each side of a cell counting chamber slide. The cell density and total cell numbers were assessed afterward.
16. Repeat steps 14-15 with the transferred media from step 12.

### Results

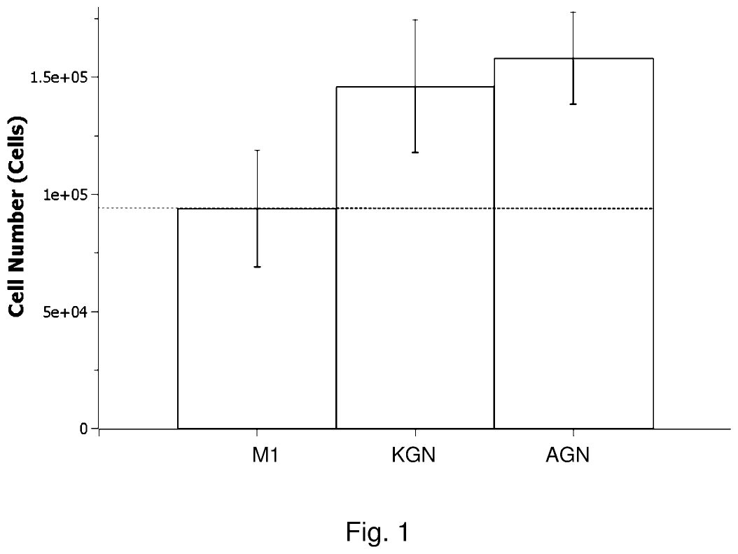

In this example, culture media with about 300 ug/mL KGN or about 300 ug/mL AGN as the first component and BSA and TGF-b1 as the second component, and the positive control (M1) were assessed for their effects on cell numbers and summarized in the results. Each sample was run in duplicates and the average total cell numbers were compared with the positive control. The cells adhered in the well plates were counted. The percentage (%) proliferation increase was calculated using the equation {[(cell number in sample)−(cell number in M1)]/(cell number in M1)}*100%. The results are shown in FIG. 1 and summarized in Table 6. In this experiment, cell viability was not assessed as the Trypsin/EDTA was not neutralized with Trypsin Neutralizer Solution.

Now referring to FIG. 1, a plot showing Porcine Skeletal Muscle cell growth under the stimulation of different sample extracts provided at about 300 ug/mL at the 56-hour time point (i.e., Samples A2 and B2, respectively, according to Table 5). The percentage (%) proliferation increase normalized to controls are tabulated in Table 6. The results showed that A2 (KGN at about 300 ug/mL) and B2 (AGN at about 300 ug/mL) in the presence of reduced final concentrations of about 1.7 ng/ml TGF-b1 and about 5.85 mg/mL BSA as the second component outperformed the positive control (which contains much higher levels of FGF-2 (about 34 ng/mL) in the M1 Media). After about 56 hours, B2 (AGN at about 300 ug/mL) reached a cell number of about 1.58×10⁵ cells while A2 (KGN at about 300 ug/mL) reached about 1.46×10⁵ cells. This is approximately 50% more than the final number of cells in the positive control (M1) with FGF-2 (with about 9.38×10⁴ cells). Additionally, serum was not used in these test groups, which demonstrates that example additive compositions containing first components such as AGN and/or KGN are effective replacements of serums in preparing serum-free (or substantially serum-free or reduced-serum) culture medium for culturing cells such as porcine skeletal muscle cells. Similar experiments can be further conducted for the other sample extracts such as Ganoderma lucidum, Coriolus versicolor, Edible Bird's Nest, Ashwaganda, and/or Cordyceps sinensis to show the unexpected technical effects of these natural substances in preparing sample extracts for example additive compositions for serum-free (or substantially serum-free or reduced-serum) culture medium for culturing cells such as myoblasts or satellite cells located in the skeletal muscle cell mix. Table 6 summarized the cell proliferation increase (%) of culture medium containing sample extract KGN and AGN in 56 hours.

TABLE 6
Cell proliferation increase (%) in 56 hours

| Sample extract | Sample | % Proliferation Increase |
|---|---|---|
| concentration | Extract | (Normalized to M1 Control) |
| 300 ug/mL | KGN | 55.65% |
| | AGN | 68.44% |

The results in Table 6 showed that culture media containing basal medium DMEM, and example additive composition containing first component of KGN, or AGN at about 300 ug/ml and a second component of about 5.85 mg/mL BSA, and about 1.7 ng/ml TGF-b1 in the final concentration had superior % proliferation increases (about 55.22% and 68.44%, respectively) at about 56 hours. Results indicated that these sample extracts (AGN and KGN) achieved surprising effects in cell proliferation for skeletal muscle cells. This example demonstrated that samples extracts can stimulate cell growth in a manner that exceeds even that of FGF-2 supplemented culture medium.

### Example 3

In this example, the effects on the cell proliferation of bovine skeletal muscle cells using the example additive compositions and culture medium compositions was demonstrated. In this example, the culture media were tested with bovine skeletal muscles cells. The culture media are also applicable to both fibroblast and skeletal muscle satellite cells.

Materials used in this example include:
Cell line: Bovine Skeletal Muscle Cells (COW-iCell-S005) (Cellverse Bioscience Technology Co., Ltd.)
Basal medium: Dulbecco's Modified Eagle Medium/Nutrient Mixture F-12 (DMEM: F12) (Thermofisher, 21041-025)
Second component: Fetal Bovine Serum (ScienCell, 0500)
Other reagents: 0.25% Trypsin/EDTA (Gibco, 25200-056), CyQuant MTT (Invitrogen, V13154), Trypsin Neutralizer Solution (Gibco, R-002-100), Gelatin (Sigma, G1890)
Sample Extracts: Bird's Nest Extract (Hunan New Mstar Biotechnology Co., Ltd, Batch MS230404), Korean Ginseng (Xi'an Demeter Biotech Co., Ltd, Batch DMTB20230106), Panax Ginseng extract (Xi'an Demeter Biotech Co., Ltd, Batch DMTB230115), Ganoderma lucidum (Xi'an Demeter Biotech Co., Ltd, Batch 2022 Oct. 12), Ashwagandha Root Extract (Xi'an Demeter Biotech Co., Ltd, Batch DMTB230320), Cordyceps sinensis Yarsagumba Polysaccharides 10% (Xi'an Demeter Biotech Co., Ltd, Batch DMTB230223), and Yunzhi Essence Capsules (Vita Green Pharmaceuticals (HK) Ltd., Batch HKNY2301A).

In this example, the steps below were carried out:
1. Stock solutions of extracts at a concentration of about 1.6 mg/mL dissolved in DI water were prepared.
2. Sterile filtration via a 0.22 um filter was performed for each extract, which was then stored in about 4° C.
3. Upon receiving bovine skeletal muscle cells, the cells were detached in 0.25% Trypsin/EDTA and neutralized in Trypsin Neutralizer Solution (also referred to as “neutralizing agent”).
4. The detached cells were centrifuged for 5 minutes at 300 g.
5. The supernatant was aspirated, followed by resuspending the cell pellet in DMEM: F12+10% FBS.
6. The cell mix was then pre-plated in an uncoated T-flask (ideally T75 flask) for 40-60 minutes, before transferring into a 2% gelatin coated T-flask (ideally T75 flask).
7. Fresh media (DMEM: F12+10% FBS) was then added to the uncoated flask, which contained the purified bovine skeletal muscle fibroblast cell. The coated flask contained purified bovine skeletal muscle cells.
8. Spent media was replaced with fresh media every 48 hours, until cells had reached 80% confluence.
9. Upon reaching confluence, 4 mL of 0.25% Trypsin/EDTA was added into the flask for 5 minutes, with occasional gentle tapping of the flasks.
10. An equal amount of Trypsin Neutralizer Solution was added to the flask and the cell mixture was centrifuged for 5 minutes at 300 g.
11. Supernatant was removed from the centrifuged cell mixture, and the cell pellet was resuspended in 1 mL fresh media.
12. Cell counts with trypan blue were performed.
13. Fresh media was added to the cell suspension until a cell density of 1e5 cells/mL was achieved.
14. 100 uL of cell suspension was then added to each well of a 96 well tissue culture plate, such that there were 10,000 cells per well.
15. Cells were then incubated at about 37° C.+5% carbon dioxide (CO₂) overnight.
16. The next day, media from the wells were removed and about 20 uL of stock extracts were added to each of the respective wells. About 180 uL of fresh DMEM/F12+about 2% FBS was added afterwards to all wells.
17. The cells were then incubated at about 37° C.+5% CO₂ for about 48 hours.
18. After about 48 hours, MTT was performed to assess cell proliferation. The rapid protocol was selected; absorbance readings at 650 nm were subtracted from readings at 570 nm to account for background signals from the wells/media.

The final concentrations of each culture medium with Korean Ginseng (Extract 1.3), Panax ginseng Extract (Extract 2.1), Ganoderma lucidum (Extract 6.1), Ashwagandha (Extract 7.1), Cordyceps sinensis (Polysaccharide Enriched) (Extract 8), Bird's Nest Extract (Extract BN), and Coriolus versicolor Extract (Extract YZ) are shown in Table 7.

TABLE 7
Final concentrations of culture medium compositions

| | Culture medium composition | | |
|---|---|---|---|
| | Additive composition | | |
| Sample Extract | First | Second | Basal |
| Name | Component | Component | medium |
| Extract 1.3 | 160 ug/mL Korean Ginseng | 2% FBS | DMEM:F12 |
| Extract 2.1 | 160 ug/mL Panax Ginseng | 2% FBS | DMEM:F12 |
| | Extract | | |
| Extract 6.1 | 160 ug/mL GL | 2% FBS | DMEM:F12 |
| Extract 7.1 | 160 ug/mL ASH | 2% FBS | DMEM:F12 |
| Extract 8 | 160 ug/mL CS | 2% FBS | DMEM:F12 |
| Extract BN | 160 ug/mL BN | 2% FBS | DMEM:F12 |
| Extract YZ | 160 ug/mL YZ | 2% FBS | DMEM:F12 |
| Negative | — | — | DMEM:F12 |
| control | | | |

### Results

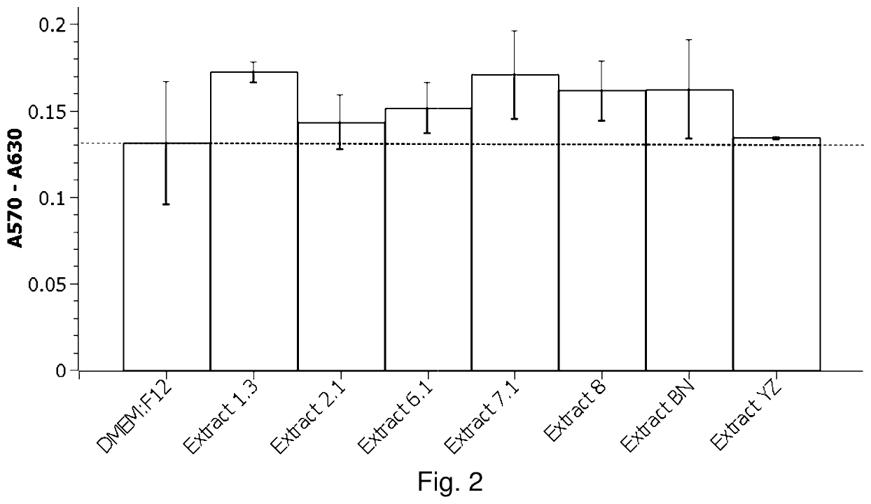

Different extracts at concentrations of about 160 ug/mL were used to culture Bovine Skeletal Muscle Fibroblasts to investigate its effectiveness in the presence of reduced serum. MTT absorbance, which is proportional to cell number, was compared with the control and expressed in percentages, as shown in FIG. 2 and summarized in Table 8. The % of proliferation increase normalized to negative control is calculated as follows:

$$\% of proliferation increase = \{ [ ( MTT absorbance in samples ) - ( MTT absorbance in negative control with DMEM : 12 ) ] / ( MTT absorbance in negative control with DMEM : 12 ) \} * 100 \%$$

Now referring to FIG. 2, a plot showing the cell growth under the stimulation of the following example additive compositions containing extracts (provided at about 160 ug/mL) at the 48-hour time point. As shown in the following figure, the extracts can exhibit various proliferative activities to the tested fibroblast cells. Additionally, cells were seeded at about 1×10⁴ cells/well (of a 96 well-plate) and were near 100% confluent. Reduced cell seeding densities may be required for these extracts to stimulate fibroblast cells, as high densities would cause contact inhibition. Regardless, these extracts demonstrate a beneficial effect on fibroblasts which can be an interesting avenue for the manufacturing of cells.

TABLE 8
Cell proliferation increase (%) in 48 hours

| | | % Proliferation |
|---|---|---|
| Sample extract | | Increase (Normalized |
| Concentration | Sample extract | to negative control) |
| 160 ug/mL | Extract 1.3 | 31.23% |
| | (Korean Ginseng) | |
| | Extract 2.1 | 9.06% |
| | (Panax ginseng Extract) | |
| | Extract 6.1 | 15.47% |
| | (Ganoderma lucidum) | |
| | Extract 7.1 | 30.02% |
| | (Ashwagandha) | |
| | Extract 8 | 23.08% |
| | (Cordyceps sinensis | |
| | (Polysaccharide | |
| | Enriched)) | |
| | Extract BN | 23.62% |
| | (Bird's Nest Extract) | |
| | Extract YZ | 2.23% |
| | (Coriolus versicolor | |
| | Extract) | |
| | Negative control | 0.00% |

The results in Table 8 showed the respective results with culture media containing basal medium DMEM: F12, and example additive composition containing first component of Korean Ginseng (Extract 1.3), Panax ginseng Extract (Extract 2.1), Ganoderma lucidum (Extract 6.1), Ashwagandha (Extract 7.1), Cordyceps sinensis (Polysaccharide Enriched) (Extract 8), Bird's Nest Extract (Extract BN), or Coriolus versicolor Extract (Extract YZ), respectively, at about 160 ug/ml and a second component of about 2% FBS in the final concentration, with the top performing extracts being Korean Ginseng, Ashwagandha, Bird's Nest, and Cordyceps sinensis having superior % proliferation increases at 56 hours of 31.23%, 30.02%, 23.62% and 23.08%, respectively. Results indicated that these sample extracts (KGN, BN, LZ, YZ, ASH, CS) have surprising effects in cell proliferation for muscle cells under conditions where the concentration of serum is significantly reduced (2% FBS).

### Example 4

In this example, the effects on the proliferation of L6 Myoblasts using culture medium compositions with no second component was demonstrated.

Materials used in this example are similar or the same as those described in the Example 1, unless otherwise specified. For the sake of clarity, the list of materials is not reproduced herein.

In this example, the steps below are carried out:
1. Stock solutions of extracts at a concentration of 1.6 mg/mL dissolved in DI water were prepared.
2. Sterile filtration via a 0.22 um filter was performed for each extract, which was then stored in 4° C.
3. L6 myoblasts were thawed from liquid nitrogen, diluted with DMEM media, centrifuged, and the pellet was resuspended in DMEM+10% FBS to a cell density of 3×10⁴ cells/mL.
4. 500 uL of cells were added to each well of a 24-well plate (2% gelatin coated) such that there were 1.5×10⁴ cells per well.
5. Cells were then incubated at 37° C.+5% CO₂ overnight.
6. The next day, media from the wells were removed and cells were washed twice with PBS.
7. DMEM with Bird's Nest Extract (Hunan New Mstar Biotechnology Co., Ltd, Batch MS230404) (Extract BN) or Panax ginseng Extract (Xi'an Demeter Biotech Co., Ltd, Batch DMTB230115) (Extract 2.1) were added to the well plate such that the final volume per well was 500 uL. The final concentrations of the compositions are shown in Table 9.

TABLE 9
Final concentrations of culture medium
compositions with no second component

| | Culture medium composition | | |
|---|---|---|---|
| | Additive composition | | |
| | First | Second | Basal |
| Sample | Component | Component | medium |
| Panax | 1 ug/mL Extract 2.1 | — | DMEM |
| Ginseng | 5 ug/mL Extract 2.1 | — | DMEM |
| Extract | 20 ug/mL Extract 2.1 | — | DMEM |
| | 40 ug/mL Extract 2.1 | — | DMEM |
| | 80 ug/mL Extract 2.1 | — | DMEM |
| Bird's Nest | 1 ug/mL Extract BN | — | DMEM |
| Extract | 5 ug/mL Extract BN | — | DMEM |
| | 20 ug/mL Extract BN | — | DMEM |
| | 40 ug/mL Extract BN | — | DMEM |
| | 80 ug/mL Extract BN | — | DMEM |
| Positive | — | 10% FBS | DMEM |
| control 1 | | | |
| Positive | — | 5 mg/mL BSA | DMEM |
| control 2 | | | |
| Negative | — | — | DMEM |
| Control | | | |

8. The cells are then incubated at about 37° C.+5% CO₂ for about 48 hours.
9. After 48 h, culture media is removed and stored in a tube, and the cells were washed with about 0.1 mL of PBS.
10. About 0.3 mL Trypsin/EDTA was then added and incubated at about 37° C. with gentle tapping occasionally. Once cells are completely detached, the media stored from step 9 was added back.
11. Cell counting was performed with a hemacytometer for analysis.

### Results

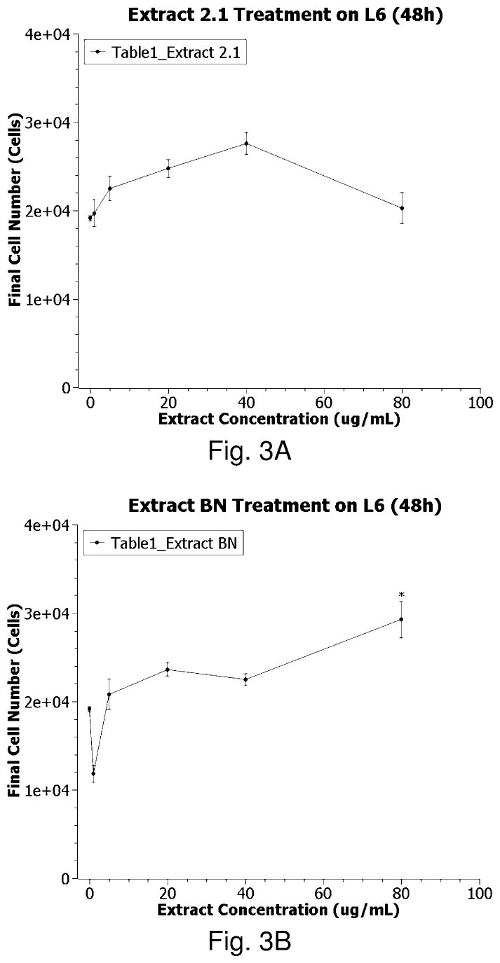

Final cell numbers after 48 h of treatment were assessed, and the results are shown in Table 10 and FIGS. 3A-3B.

TABLE 10
Cell proliferation increase (%) in 48 hours

| | |
|---|---|
| Final | % Proliferation Increase (Normalized |
| Concentration | to negative control) |

| (ug/mL) | Extract 2.1 | Extract BN |
|---|---|---|
| 0 | 0.00% | 0.00% |
| 1 | 2.94% | −38.24% |
| 5 | 17.65% | 8.82% |
| 20 | 29.41% | 23.53% |
| 40 | 44.12% | 17.65% |
| 80 | 5.88% | 52.94% |

Now referring to FIGS. 3A-3B, the final cell numbers of L6 myoblast cells treated for 48 hours in culture media with varying concentrations of Panax Ginseng Extract (Extract 2.1) and Bird's Nest Extract (Extract BN) according to Table 9 are shown. For the culture media with example additive compositions containing first component of various final concentrations of Panax Ginseng Extract (Extract 2.1) (and without any second component), it was observed that all culture media with example additive compositions containing first component of various final concentrations of Panax Ginseng Extract at 1-80 ug/ml had a general increase in % proliferation. It is also observed that there was an initial increase in cell number as dosages increased from 1 to 40 ug/ml. Final cell number peaked at about 40 ug/mL Extract 2.1, with a 44.12% proliferation increase compared to the negative control, according to Table 10. This was accompanied by a dramatic decrease in final cell number with 80 ug/mL Extract 2.1. This shows the surprising effect of Extract 2.1 at a final concentration of 40 ug/mL on cell proliferation given that no serum, growth factors or hormones were present in the culture medium.

For the culture media with example additive compositions containing first component of various final concentrations (1-80 ug/ml) of Bird's Nest (Extract BN) (and without any second component), higher final cell number was observed as the concentration of Extract BN increased from 5-80 ug/ml-, which is indicative of a positive proliferative effect of Extract BN in myoblast cells. In the presence of about 80 ug/mL Extract BN, a 52.94% proliferation increase was achieved, accordingly to Table 10. This shows the surprising effect of Extract BN on cell proliferation given that no serum, growth factors or hormones were present in the culture medium.

It was noted the final cell number of DMEM with BSA vs DMEM without BSA is about 1.35×10⁴ and about 1.91×10⁴ cells, respectively, according to Table 9. This indicates that 5 mg/mL BSA may be a concentration that is detrimental to cells, and BSA should be eliminated or reduced.

In summary, results showed that there is a surprising beneficial effect when cells are treated with sample additive composition containing bird's nest (Extract BN) and ginseng (Extract 2.1), as most test groups had a higher cell proliferation rate compared to when cells are not treated with sample extracts.

This example demonstrates that, even in serum-free culture medium, L6 cells treated with BN and Panax ginseng extracts can still outperform the non-treated controls in stimulating cell proliferation.

The examples disclosed herein show that various sample extracts, at various concentrations and timepoints, can have different cellular effects such as cell proliferation and differentiation.

In some embodiments, different types of extracts, at different concentrations, different culture timepoints and different suppliers of extracts will affect different cell types (e.g. Fibroblasts, Skeletal Muscle Satellites, Skeletal Muscle Myoblasts, Adipocytes etc.) proliferation, differentiation, and gene expressions (e.g. Pax7, MyoD, MHC).

In some embodiments, increased dosages and treatment time corresponds to changes in cell number. In some embodiments, a combination of extracts in the additive composition displays antagonistic, additive or synergistic effects.

In some embodiments, after assessing the relationships between various extract factors (concentration, timepoints, etc.) and cell growth/differentiation, experimental methods utilizing multivariate statistics can be utilized to create a model displaying the synergistic activities between extracts. The optimal extract combination that results in various cellular effects (cell growth, differentiation, long-term passage, stemness) can be chosen by using this model.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

For example, other cell lines for animal tissues (e.g. muscle, fat, fibroblast, vascular cells, pericytes, endothelial cells, myoblast cells, satellite cells, fat cells, adipose-derived stem cells, smooth muscle cells, skin cells, tendon, liver, brain, bone, heart, kidney, stem cells, and combination thereof) may be used.

For example, other media of choice and additives can be used.

For example, water is used as a solvent to extract the samples to form an aqueous solution, but other solvents such as DMSO, methanol, ethanol etc. can be used.

For example, the sample extracts can be commercially available or prepared from natural substances.

### NUMBERED EMBODIMENTS

### Numbered Embodiments 1

Embodiment 1. A culture medium additive composition, comprising at least one sample extract prepared from at least one natural substance, wherein the at least one natural substance is a sample selected from a group consisting of bird's nests, ginsengs, Ganoderma lucidum, versicolor mushrooms, Cordyceps sinensis, Ashwagandha, and combination thereof.

Embodiment 2. The composition of any one of the preceding embodiments, wherein the ginseng is American Ginseng or Korean Ginseng.

Embodiment 3. The composition of any one of the preceding embodiments, wherein the composition substantially does not comprise one or more exogenous growth factors or hormones, or combination thereof.

Embodiment 4. The composition of any one of the preceding embodiments, wherein the at least one sample extract is bird's nest.

Embodiment 5. The composition of any one of the preceding embodiments, wherein the at least one sample extract is American Ginseng.

Embodiment 6. The composition of any one of the preceding embodiments, wherein the at least one sample extract is Korean Ginseng.

Embodiment 7. The composition of any one of the preceding embodiments, wherein the at least one sample extract is Ganoderma lucidum.

Embodiment 8. The composition of any one of the preceding embodiments, wherein the at least one sample extract is versicolor mushroom.

Embodiment 9. The composition of any one of the preceding embodiments, wherein the at least one sample extract is Cordyceps sinensis.

Embodiment 10. The composition of any one of the preceding embodiments, wherein the at least one sample extract is Ashwaganda.

Embodiment 11. The composition of any one of the preceding embodiments, wherein the Ashwaganda is derived from a root or a whole plant.

Embodiment 12. The composition of any one of the preceding embodiments, wherein the sample extract is prepared from a solution of a filtrate or a supernatant.

Embodiment 13. The composition of any one of the preceding embodiments, wherein the composition essentially consists of at least one sample extract prepared from at least one sample selected from a group consisting of bird's nests, ginsengs, Ganoderma lucidum, versicolor mushrooms, Cordyceps sinensis, Ashwagandha, and combination thereof.

Embodiment 14. The composition of any one of the preceding embodiments, wherein the at least one sample extract has a concentration of 0.001 ug/mL-1000 mg/mL.

Embodiment 15. The composition of any one of the preceding embodiments, wherein the at least one sample extract has a concentration of 20 mg/mL-100 mg/mL.

Embodiment 16. A culture medium, comprising a basal medium or a minimal medium and the culture media additive composition as described in any one of the preceding embodiments.

Embodiment 17. The culture medium of any one of the preceding embodiments, wherein the culture medium substantially does not comprise one or more exogenous growth factors or hormones, or combination thereof.

Embodiment 18. The culture medium of any one of the preceding embodiments, wherein the culture medium further comprises one or more exogenous growth factors or hormones, or combination thereof.

Embodiment 19. The culture media of any one of the preceding embodiments, wherein the basal medium is DMEM and/or F12.

Embodiment 20. The culture media of any one of the preceding embodiments, further comprising FBS at a final concentration of 0.01-20%.

Embodiment 21. A method of culturing cells, comprising the following steps: (i) providing a culture medium comprising a basal medium and at least one sample extract prepared from at least one sample consisting of bird's nests, ginsengs, Ganoderma lucidum, versicolor mushrooms, Cordyceps sinensis, Ashwagandha, and combination thereof; and (ii) treating and culturing cells with the at least one natural substance.

Embodiment 22. The method of any one of the preceding embodiments, wherein the method further comprises step of: (1) treating a cell line with a detachment agent to form a cell suspension, prior to step (ii).

Embodiment 23. The method of any one of the preceding embodiments, wherein the method further comprises step of: (2) neutralizing the cell suspension with a neutralizing agent to obtain a neutralized cell suspension, prior to step (ii).

Embodiment 24. The method of any one of the preceding embodiments, wherein the method further comprises step of: (3) obtaining a cell pellet from the neutralized cell suspension and resuspending the cell pellet in the culture medium.

Embodiment 25. The method of any one of the preceding embodiments, wherein the sample extract is prepared by the following steps: (a) preparing at least one powder of the at least one sample; (b) dissolving the at least one powder to form at least one sample solution; (c) purifying the at least one sample solution to obtain the at least one sample extract.

Embodiment 26. The method of any one of the preceding embodiments, wherein the purifying the at least one sample solution in step (c) is filtering and/or centrifuging the at least one sample solution.

Embodiment 27. The method of any one of the preceding embodiments, wherein the culture medium has a final sample extract concentration of 0.001 ug/mL-1000 mg/mL.

Embodiment 28. The method of any one of the preceding embodiments, wherein the culture medium has a final sample extract concentration of 0.1 mg/mL-0.5 mg/mL.

Embodiment 29. The method of any one of the preceding embodiments, wherein the culturing cells are for producing animal tissues.

Embodiment 30. The method of any one of the preceding embodiments, wherein the animal tissues are human tissues.

Embodiment 31. The method of any one of the preceding embodiments, wherein the culturing cells are for producing cultured-meat.

### Numbered Embodiments 2

Embodiment 1. An additive composition for a culture medium, comprising a first component comprising at least one sample extract prepared from at least one natural substance, wherein the at least one natural substance is selected from a group consisting of bird's nest (BN), ginseng, Ling Zhi, versicolor mushroom, Cordyceps sinensis (CS), Ashwagandha (ASH), and combinations thereof.

Embodiment 2. The composition of embodiment 1, wherein the ginseng is Panax quinquefolius, Panax ginseng, and combinations thereof.

Embodiment 3. The composition of any one of the preceding embodiments, wherein the versicolor mushroom is Yun Zhi (YZ).

Embodiment 4. The composition of any one of the preceding embodiments, wherein the Ling Zhi is Ganoderma lucidum (GL).

Embodiment 5. The composition of any one of the preceding embodiments, wherein the versicolor mushroom is Coriolus versicolor.

Embodiment 6. The composition of any one of the preceding embodiments, wherein the first component substantially does not comprise one or more exogenous serums, growth factors or hormones, or combinations thereof.

Embodiment 7. The composition of any one of the preceding embodiments, wherein the culture medium is for culturing animal cells.

Embodiment 8. The composition of any one of the preceding embodiments, wherein the culture medium is for culturing mammalian, avian, crustaceans or fish cells.

Embodiment 9. The composition of any one of the preceding embodiments, wherein the culture medium is for culturing fibroblasts, vascular cells, pericytes, endothelial cells, myoblast cells, skeletal muscle satellite cells, muscle cells, smooth muscle cells, fat cells, adipose-derived stem cells, skin cells, tendon, liver, brain, bone, heart, kidney, stem cells, or combination thereof.

Embodiment 10. The composition of any one of the preceding embodiments, wherein the culture medium is for culturing fibroblasts or myoblasts.

Embodiment 11. The composition of any one of the preceding embodiments, wherein the individual sample extract is prepared from a solution of a filtrate or a supernatant of the individual natural substance.

Embodiment 12. The composition of any one of the preceding embodiments, wherein the first component essentially consists of at least one sample extract prepared from at least one natural substance, wherein the at least one natural substance is selected from a group consisting of bird's nests, ginsengs, Ganoderma lucidum, versicolor mushrooms, Cordyceps sinensis, Ashwagandha, and combination thereof.

Embodiment 13. The composition of any one of the preceding embodiments, wherein the individual sample extract has a final concentration of about 0.01 ng/mL-1000 mg/mL in the culture medium.

Embodiment 14. The composition of any one of the preceding embodiments, wherein the individual sample extract has a final concentration of about less than 1000 or 500 ug/mL in the culture medium.

Embodiment 15. The composition of any one of the preceding embodiments, further comprising a second component comprising at least one serum, growth factor or hormone, amino acid, vitamin, salt, minerals, glucose, buffering agent, or combinations thereof.

Embodiment 16. The composition of any one of the preceding embodiments, wherein the second component further comprises fetal bovine serum (FBS) at a final concentration of less than about 20%, 10%, 5% or 2% in the culture medium.

Embodiment 17. The composition of any one of the preceding embodiments, wherein the second component further comprises bovine serum albumin (BSA) at a final concentration of about 0.01 ng/mL-10 mg/mL in the culture medium.

Embodiment 18. The composition of any one of the preceding embodiments, wherein the second component further comprises bovine serum albumin (BSA) at a final concentration of less than about 10, 9, 8, 7, 6, 5, 4, 3, 2, or 1 mg/mL in the culture medium.

Embodiment 19. The composition of any one of the preceding embodiments, wherein the at least one growth factor or hormone is FGF-2, TGF-b1, vascular endothelial growth factor (VEGF), insulin-like growth factor (IGF), Insulin, EGF, platelet-derived growth factor (PDGF), interleukin 6 (IL-6) or combinations thereof.

Embodiment 20. The composition of any one of the preceding embodiments, wherein the second component further comprises TGF-b1 is present at a final concentration of less than about 10, 9, 8, 7, 6, 5, 4, 3, 2, 1 or 0.5 ng/ml in the culture medium.

Embodiment 21. The composition of any one of the preceding embodiments, wherein the second component further comprises fetal bovine serum (FBS), fetal calf serum, bovine serum albumin (BSA), or combinations thereof.

Embodiment 22. An additive composition for a culture medium for culturing myoblasts, the additive composition comprises: a first component, comprising at least one sample extract prepared from at least one natural substance, wherein the at least one natural substance is selected from a group consisting of bird's nest (BN), American Ginseng (AGN) and Korean Ginseng (KGN), and combination thereof, wherein individual sample extract has a final concentration of about 1-500 ug/mL in the culture medium.

Embodiment 23. The composition of any one of the preceding embodiments, further comprising a second component comprising FBS having a final concentration of about 2-5% in the culture medium.

Embodiment 24. An additive composition for a culture medium for culturing porcine skeletal muscle cells, the additive composition comprises: a first component, comprising at least one sample extract prepared from at least one natural substance, wherein the at least one natural substance is selected from a group consisting of bird's nest (BN), American Ginseng (AGN) and Korean Ginseng (KGN), and combination thereof, wherein individual sample extract has a final concentration of about 1-500 ug/mL in the culture medium.

Embodiment 25. The composition of any one of the preceding embodiments, further comprising a second component comprising FBS having a final concentration of about 2-5% in the culture medium.

Embodiment 26. The composition of any one of the preceding embodiments, further comprising a second component comprising BSA having a final concentration of about 1-10 mg/ml and optionally TGF-b1 having a final concentration of about 0.5-10 ng/ml in the culture medium.

Embodiment 27. An additive composition for a culture medium for culturing bovine skeletal muscle cells, the additive composition comprises: a first component, comprising at least one sample extract prepared from at least one natural substance, wherein the at least one natural substance is selected from a group consisting of bird's nest (BN), ginseng, Ling Zhi, versicolor mushroom, Cordyceps sinensis (CS), Ashwagandha (ASH), and combinations thereof, wherein individual sample extract has a final concentration of about 1-500 ug/mL in the culture medium.

Embodiment 28. The composition of any one of the preceding embodiments, further comprising a second component comprising FBS having a final concentration of about 2-5% in the culture medium.

Embodiment 29. A culture medium, comprising a basal medium or a minimal medium and the culture medium additive composition as described in any one of the preceding embodiments.

Embodiment 30. The culture medium of any one of the preceding embodiments, wherein the culture medium substantially does not comprise or comprise a reduced amount of one or more exogenous serums, growth factors or hormones, or combinations thereof.

Embodiment 31. The culture medium of any one of the preceding embodiments, wherein the basal medium or minimal medium is Dulbecco's Modified Eagle Medium (DMEM), Ham's F12 medium (F12), Dulbecco's Modified Eagle Medium F12 (DMEM: F12), Ham's F10 medium (F10), Roswell Park Memorial Institute 1640 Medium (RPMI 1640), Minimum Essential Medium (MEM), serum free medium (SFM) or combination thereof.

Embodiment 32. A method of culturing cells, comprising the following steps: (i) providing a basal medium or minimal medium and the culture medium additive composition as described in any one of the preceding embodiments to form a culture medium; and (ii) treating and culturing cells with the culture medium.

Embodiment 33. The method of any one of the preceding embodiments, wherein the method further comprises a step of: (1) treating a cell line with a detachment agent to form a cell suspension.

Embodiment 34. The method of any one of the preceding embodiments, wherein the method further comprises steps of: (2) neutralizing the cell suspension with a neutralizing agent to obtain a neutralized cell suspension.

Embodiment 35. The method of any one of the preceding embodiments, wherein the method further comprises step of: (3) obtaining a cell pellet from the neutralized cell suspension and resuspending the cell pellet in the culture medium.

Embodiment 36. The method of any one of the preceding embodiments, wherein the sample extract is prepared by one or more of the following steps: (a) preparing at least one powder of the at least one natural substance; (b) dissolving the at least one powder to form at least one sample solution; (c) purifying the at least one sample solution to obtain the at least one sample extract.

Embodiment 37. The method of any one of the preceding embodiments, wherein the purifying the at least one sample solution in step (c) is filtering and/or centrifuging the at least one sample solution.

Embodiment 38. The method of any one of the preceding embodiments, wherein the culturing cells are for producing animal tissues.

Embodiment 39. The method of any one of the preceding embodiments, wherein the animal tissues are mammalian, avian, crustaceans or fish tissues.

Embodiment 40. The method of any one of the preceding embodiments, wherein the culturing cells are for producing cultured-meat.

Embodiment 41. The method of any one of the preceding embodiments, further comprising a step of incubating the cells in a CO₂ incubator.

Embodiment 42. The method of any one of the preceding embodiments, wherein the cell line is derived from mammals, birds, crustaceans or fish.

Embodiment 43. The method of any one of the preceding embodiments, wherein the cell line is derived from fibroblasts, vascular cells, pericytes, endothelial cells, myoblast cells, satellite cells, skeletal muscle cells, muscle cells, smooth muscle cells, fat cells, adipose-derived stem cells, skin cells, tendon, liver, brain, bone, heart, kidney, stem cells, and combination thereof.

1. An additive composition for a culture medium, comprising a first component comprising at least one sample extract prepared from at least one natural substance, wherein the at least one natural substance is selected from a group consisting of bird's nest (BN), ginseng, Ling Zhi, versicolor mushroom, Cordyceps sinensis (CS), Ashwagandha (ASH), and combinations thereof.

2. The composition of claim 1, wherein the ginseng is Panax quinquefolius, Panax ginseng, or combinations thereof;
wherein the versicolor mushroom is Yun Zhi (YZ);
wherein the Ling Zhi is Ganoderma lucidum (GL); and
wherein the versicolor mushroom is Coriolus versicolor.

3-5. (canceled)

6. The composition of claim 1, wherein the first component substantially does not comprise one or more exogenous serums, growth factors or hormones, or combinations thereof.

7. The composition of claim 1, wherein the culture medium is for culturing animal cells.

8. (canceled)

9. The composition of claim 1, wherein the culture medium is for culturing fibroblasts, vascular cells, pericytes, endothelial cells, myoblast cells, skeletal muscle satellite cells, muscle cells, smooth muscle cells, fat cells, adipose-derived stem cells, skin cells, tendon, liver, brain, bone, heart, kidney, stem cells, or combinations thereof.

10-12. (canceled)

13. The composition of claim 1, wherein an individual sample extract has a final concentration of about 0.01 ng/ml-1000 mg/mL in the culture medium.

14. (canceled)

15. The composition of claim 1, further comprising a second component comprising at least one serum, growth factor or hormone, amino acid, vitamin, salt, minerals, glucose, buffering agent, or combinations thereof.

16. The composition of claim 15, wherein the second component further comprises fetal bovine serum (FBS) at a final concentration of less than about 20%, 10%, 5% or 2% in the culture medium; or wherein the second component further comprises bovine serum albumin (BSA) at a final concentration of about 0.01 ng/ml-10 mg/mL in the culture medium.

17-18. (canceled)

19. The composition of claim 15, wherein the at least one growth factor or hormone is FGF-2, TGF-b1, vascular endothelial growth factor (VEGF), insulin-like growth factor (IGF), Insulin, EGF, platelet-derived growth factor (PDGF), interleukin 6 (IL-6) or combinations thereof.

20. (canceled)

21. The composition of claim 15, wherein the second component further comprises fetal bovine serum (FBS), fetal calf serum, bovine serum albumin (BSA), or combinations thereof.

22. The composition of claim 1, wherein the composition is for culturing myoblasts, and wherein the additive composition comprises:
a first component, comprising at least one sample extract prepared from at least one natural substance, wherein the at least one natural substance is selected from a group consisting of bird's nest (BN), American Ginseng (AGN), Korean Ginseng (KGN), and combinations thereof, wherein an individual sample extract has a final concentration of about 1-500 ug/mL in the culture medium; and wherein optionally the additive composition further comprises a second component comprising FBS having a final concentration of about 2-5% in the culture medium.

23. (canceled)

24. The additive composition of claim 1, wherein the composition is for culturing porcine skeletal muscle cells, and wherein the additive composition comprises:
a first component, comprising at least one sample extract prepared from at least one natural substance, wherein the at least one natural substance is selected from a group consisting of bird's nest (BN), American Ginseng (AGN), Korean Ginseng (KGN), and combinations thereof, wherein an individual sample extract has a final concentration of about 1-500 ug/mL in the culture medium; and wherein optionally the additive composition further comprises a second component comprising FBS having a final concentration of about 2-5% in the culture medium.

25-26. (canceled)

27. The additive composition of claim 1, wherein the composition is for culturing bovine skeletal muscle cells, and wherein the additive composition comprises:
a first component, comprising at least one sample extract prepared from at least one natural substance, wherein the at least one natural substance is selected from a group consisting of bird's nest (BN), ginseng, Ling Zhi, versicolor mushroom, Cordyceps sinensis (CS), Ashwagandha (ASH), and combinations thereof, wherein an individual sample extract has a final concentration of about 1-500 ug/mL in the culture medium; and wherein optionally the additive composition further comprises a second component comprising FBS having a final concentration of about 2-5% in the culture medium.

28. (canceled)

29. A culture medium, comprising a basal medium or a minimal medium and the culture medium additive composition as claimed in claim 1.

30. The culture medium of claim 29, wherein the culture medium substantially does not comprise or comprises a reduced amount of one or more exogenous serums, growth factors or hormones, or combinations thereof;
wherein the basal medium or minimal medium is Dulbecco's Modified Eagle Medium (DMEM), Ham's F12 medium (F12), Dulbecco's Modified Eagle Medium F12 (DMEM: F12), Ham's F10 medium (F10), Roswell Park Memorial Institute 1640 Medium (RPMI 1640), Minimum Essential Medium (MEM), serum free medium (SFM), or combinations thereof.

31. (canceled)

32. A method of culturing cells, comprising the following steps:
(i) providing a basal medium or minimal medium and the culture medium additive composition as claimed in claim 1 to form a culture medium; and
(ii) treating and culturing cells with the culture medium.

33. The method of claim 32, wherein the method further comprises a step of:
(1) treating a cell line with a detachment agent to form a cell suspension.

34-35. (canceled)

36. The method of claim 32, wherein the sample extract is prepared by one or more of the following steps:
(a) preparing at least one powder of the at least one natural substance;
(b) dissolving the at least one powder to form at least one sample solution;
(c) purifying the at least one sample solution to obtain the at least one sample extract; optionally, the step (c) comprises the step of filtering and/or centrifuging the at least one sample solution.

37. (canceled)

38. The method of claim 32, wherein the culturing cells are for producing animal tissues; optionally, wherein the animal tissues are mammalian, avian, crustaceans or fish tissues; or
optionally, wherein the culturing cells are for producing cultured-meat.

39-41. (canceled)

42. The method of claim 32, wherein the cell line is derived from mammals, birds, crustaceans or fish; optionally, wherein the cell line is derived from fibroblasts, vascular cells, pericytes, endothelial cells, myoblast cells, satellite cells, skeletal muscle cells, muscle cells, smooth muscle cells, fat cells, adipose-derived stem cells, skin cells, tendon, liver, brain, bone, heart, kidney, stem cells, or combinations thereof.

43. (canceled)
